SEQUENCE LISTING (2 sequences)

SEQ ID NO: 1 - DNA, 24 bp, Artificial Sequence
  Forward primer sequence labeled with 6-fluorescein amidite (6-FMA)
tgacattttagggagctggagaca

SEQ ID NO: 2 - DNA, 20 bp, Artificial Sequence
  reverse primer sequence
acaaagtctggccataggac

(19) United States
(12) Patent Application Publication
(10) Pub. No.: US 2013/0129688 A1
(43) Pub. Date: May 23, 2013

(54) Assay for the Prediction of Therapeutic Effectiveness or Potency of Mesenchymal Stem Cells, and Methods of Using Same

(71) Applicant: Allocure, Inc., Salt Lake City, UT (US)

(72) Inventors: Robert M. Brenner, Weston, MA (US); Ali S. Mohamed, Boyds, MD (US); Dena Minning, Indialantic, FL (US)

(73) Assignee: ALLOCURE, INC., Salt Lake City, UT (US)

(21) Appl. No.: 13/671,417

(22) Filed: Nov. 7, 2012

Related U.S. Application Data
(60) Provisional application No. 61/557,616, filed on Nov. 9, 2011.

(51) Int. Cl. C12Q 1/68

(52) U.S. Cl. CPC C12Q 1/6888
USPC 424/93.7; 435/6.11; 435/366

(57) ABSTRACT

The invention relates to assays for testing the therapeutic effectiveness of mesenchymal stem cell (MSC) populations by determining the number of GT repeats in the heme oxygenase-1 (HO-1) promoter region of both alleles.

### RELATED APPLICATIONS

This application claims the benefit of priority under 35 U.S.C. §119(e) to U.S. Patent Application Ser. No. 61/557,616 filed Nov. 9, 2011, which is hereby incorporated by reference in its entirety.

### INCORPORATION-BY-REFERENCE OF SEQUENCE LISTING

The instant application contains a Sequence Listing which has been submitted via EFS-Web. The contents of the text file named “38447-506001US_ST25.txt”, which was created on Nov. 7, 2012 and is 1 KB in size, are hereby incorporated by reference in their entirety.

### FIELD OF THE INVENTION

The present invention generally relates to assays that predict the therapeutic effectiveness or potency of mesenchymal stem cells.

### BACKGROUND OF THE INVENTION

Stem cell therapy offers a promising new option for the treatment of human disease. Mesenchymal stem cells (MSCs) are cells derived from bone marrow, adipose, and/or cord blood that have the ability to differentiate into a variety of cell types under certain conditions, possess immunomodulatory properties and secrete chemokines, cytokines and growth factors (Schinkothe et al., Stem Cells Dev. 2008; 17: 199-206), together making them ideal candidate therapies of various disorders (Porada et al., Curr Stem Cell Res Ther. 2006; 1:365-9). MSCs have been shown to successfully to treat a number of conditions in animal models and are currently being evaluated in clinical trials to treat different diseases including acute kidney injury (AKI), myocardial infarction, graft versus host disease, Crohn's disease and others (Giordano et al., J Cell Physiol. 2007; 211: 27-35).

MSCs are effective in reducing kidney injury and enhancing recovery of kidney function in a variety of animal models of AKI, including an ischemia/reperfusion model, a glycerol model, as well as in cytotoxicity models such as cisplatin-induced AKI. Importantly, in these models, MSC do not or only rarely differentiate to directly contribute to kidney cell types, e.g. tubular cells, endothelial cells, or other cell types (Humphreys et al. Minerva Urol Nefrol. 2006; 58: 329-37). Instead, MSCs mediate benefit and promote kidney recovery through paracrine and potentially endocrine mechanisms via the release secreted mediators including stromal cell-derived factor-1 (SDF-1), vascular endothelial growth factor (VEGF) and other vasculotropic factors, insulin-like growth factor (IGF-1), hepatocye growth factor (HGF) (Fogel et al., Am J Physiol Renal Physiol. 2007; 292:F1626-35; Imberti et al., J Am Soc Nephrol, 2007; 18: 2921-8)), and other factors that promote organ repair. Of note, the beneficial effect of MSC has been reproduced using conditioned medium from MSCs in an animal model of AKI. (Bi et al. J Am Soc Nephrol. 2007; 18: 2486-96).

For therapeutic use of MSCs, a sufficient number of cells are needed to provide an adequate dose. Thus, in most situations, MSCs must be expanded to generate a sufficient number of cells for a therapeutic effective dose that may be frozen in order to treat patients at a clinically relevant time. The effectiveness or potency of MSCs in treating various pathologies must be confirmed when the cells are passaged, expanded, and/or frozen.

Thus, there is a need in the art to be able to determine and/or predict the therapeutic effectiveness of populations of MSCs.

### SUMMARY OF THE INVENTION

Provided herein are methods of generating populations of human MSCs, by determining the number of guanine-thymine (GT) dinucleotide repeats present in the heme oxygenase-1 (HO-1) promoter region of both alleles of human MSCs. Such methods can additionally involve the step of selecting those human MSCs having 32 or fewer (e.g., between 21 and 32) GT repeats in both alleles.

Likewise, these methods can additionally involve the step of expanding the human MSCs in a platelet lysate (PL) supplemented culture medium to generate an expanded population of human MSCs. In some embodiments, the human MSCs are expanded prior to determining the number of GT repeats present in both alleles. In other embodiments, the number of GT repeats present in both alleles is determined prior to isolating and/or expanding the human MSCs.

The invention also provides methods of assaying the therapeutic effectiveness of human MSCs for treating a pathology in a subject by obtaining (or providing) a population of MSCs and analyzing the number of GT repeats present in the HO-1 promoter region of both alleles to determine whether the MSCs have short, medium, or long alleles. The presence of two short alleles, two medium alleles, or one short allele and one medium allele indicates that the population contains MSCs that are more therapeutically effective. MSC populations from donors having one or more long alleles will be excluded from clinical use for being less therapeutically effective.

For example, the population of human MSCs can be autologous or allogeneic to the subject. The human tissue used to genotype for the number of GT repeats can be obtained (or provided) from any suitable source of genetic material, including, but not limited to a peripheral blood sample, saliva, buccal swab, a cryopreserved MSC sample, a Master Cell Bank (MCB), and/or a bone marrow sample. Those skilled in the art will recognize that it is possible to obtain or provide MSCs from an ex vivo source/sample.

The pathology to be treated may be one or more of the following: a neurological pathology (e.g., stroke), an inflammatory pathology (e.g., multi-organ failure), a renal pathology (e.g., acute kidney injury, acute renal failure, chronic renal failure, chronic kidney disease, transplant, diabetic nephropathy, and hypertensive nephropathy), a hepatic pathology, a cardiovascular pathology, a retinal pathology, a muscular pathology, a bone-related pathology, a gastrointestinal pathology, a skin related pathology and/or a metabolic pathology (e.g., diabetes).

Human MSCs suitable for use in any of the methods of the invention preferably have 32 or fewer GT repeats in both alleles of the HO-1 promoter region. For example, the human MSCs utilized may have two short alleles, two medium alleles, or one short and one medium allele wherein a short allele has ≦26 GT repeats in the HO-1 promoter region and wherein a medium allele has between 27 and 32 GT repeats in the HO-1 promoter region. MSCs containing one or more long alleles are less therapeutically effective. Therefore, ideally, the human MSCs do not have any long alleles, wherein a long allele has >32 GT repeats in the HO-1 promoter region.

As used herein, a “short” allele can have ≦26 GT repeats (e.g., between about 21 and about 26 GT repeats); a “medium” allele can have between about 27 and about 32 GT repeats; and a “long” allele can have >32 GT repeats (e.g., between about 33 and about 44 GT repeats).

The number of GT repeats in an allele can be analyzed using any suitable method known in the art, including, but not limited to DNA Fragment Length Analysis or DNA sequencing methodologies.

In another embodiment, the invention provides methods of selecting donors having therapeutically effective human MSCs for treating a pathology in a subject by (a) analyzing the number of GT repeats present in the HO-1 promoter region of both alleles in genetic material from a potential human donor to determine whether the potential donor has short, medium, or long alleles, wherein the presence of two short alleles, two medium alleles, or one short allele and one medium allele indicates that the potential donor would provide MSCs that are therapeutically effective, and (b) selecting those donors having such MSCs. By way of non-limiting example, the human donor may be a bone marrow donor, an adipose tissue donor, a cord blood tissue donor, and/or a donor of any other tissue having MSCs.

In such methods, the donor may be autologous or allogeneic to the subject. Moreover, the number of GT repeats may be analyzed from a blood sample, from a saliva sample, from a cryopreserved MSC sample, from a sample from a MCB, from a bone marrow sample, or from another suitable source of genetic material. Those skilled in the art will recognize that it is possible to obtain or provide MSCs from an ex vivo source/sample.

Similarly, the pathology may be selected from the group consisting of a neurological pathology (e.g., stroke), an inflammatory pathology (e.g., multi-organ failure), a renal pathology (e.g., acute kidney injury, acute renal failure, chronic renal failure, chronic kidney disease, transplant, diabetic nephrology, and/or hypertensive nephrology), a hepatic pathology, a cardiovascular pathology, a retinal pathology, a muscular pathology, a bone-related pathology, a gastrointestinal pathology, a skin related pathology and a metabolic pathology (e.g., diabetes).

Those skilled in the art will recognize that in these methods, a short allele has ≦26 (e.g., between about 21 and about 26) GT repeats; a medium allele has between about 27 and about 32 GT repeats; and a long allele has >32 (e.g., between about 33 and about 44) GT repeats.

Any suitable methods for analyzing the number of GT repeats can be used (e.g., DNA Fragment Length Analysis or DNA sequencing).

Also provided are methods of treating any suitable disease (e.g., an MSC-related pathology) in a subject in need thereof by (a) obtaining (or providing) a population of human MSCs; (b) analyzing the number of GT repeats present in the HO-1 promoter region of both alleles to determine whether the MSCs have short, medium, or long alleles, wherein the presence of two short alleles, two medium alleles, or one short allele and one medium allele indicates that the population contains MSCs that are therapeutically effective; and (c) administering an effective dose of the therapeutically effective MSCs to the subject, thereby treating the disease (e.g., the MSC-related pathology) in the subject.

The invention additionally provides populations of therapeutically effective MSCs for use in treating an MSC-related pathology, wherein the population of therapeutically effective MSCs is obtained by: (a) obtaining (or providing) a population of human MSCs; (b) analyzing the number of GT repeats present in the HO-1 promoter region of both alleles to determine whether the MSCs have short, medium, or long alleles, wherein the presence of two short alleles, two medium alleles, or one short allele and one medium allele indicates that the population contains MSCs that are therapeutically effective; and (c) selecting the population of therapeutically effective MSCs.

MSCs can be administered to the patient using any route of administration known in the art. By way of non-limiting example, the MSCs can be administered intra-arterially or intravenously to the patient. In some embodiments, the MSCs are administered to the patient in a biologically and physiologically compatible solution. Preferably, the solution is not enriched for pluripotent hematopoietic stem cells.

In some embodiments of the invention, an effective amount of MSCs is between about 7×10⁵ and about 7×10⁶ cells/kg.

The population of human MSCs can be autologous or allogeneic to the subject. Additionally, the MSCs can be non-transformed stem cells. The population of human MSCs can be obtained (or provided) from any suitable source, including, but not limited to, a cryopreserved sample, a MCB, a bone marrow sample, an adipose tissue sample, and/or a cord blood sample. Any potential sources of MSCs can be utilized. Those skilled in the art will recognize that it is possible to obtain or provide MSCs from an ex vivo source/sample. Moreover, the patient may be any living organisms such as humans, non-human animals (e.g., monkeys, cows, sheep, horses, pigs, cattle, goats, dogs, cats, mice, or rats), cultured cells therefrom, and transgenic species thereof.

The MSC-related pathology to be treated may be one or more of the following: a neurological pathology (e.g., stroke), an inflammatory pathology (e.g., multi-organ failure), a renal pathology (e.g., acute kidney injury, acute renal failure, chronic renal failure, chronic kidney disease, transplant, diabetic nephrology, and hypertensive nephrology), a hepatic pathology, a cardiovascular pathology, a retinal pathology, a muscular pathology, a bone-related pathology, a gastrointestinal pathology, a skin related pathology and/or a metabolic pathology (e.g., diabetes).

In these methods, a short allele has ≦26 (e.g., between about 21 and about 26) GT repeats, a medium allele has between about 27 and about 32 GT repeats, and a long allele has >32 (e.g., between about 33 and about 44) GT repeats.

In still further embodiments, the invention provides methods of treating an MSC-related pathology in a subject in need thereof by (a) analyzing the number of GT repeats present in the HO-1 promoter region of both alleles of a potential human donor to determine whether the potential donor has short, medium, or long alleles, wherein the presence of two short alleles, two medium alleles, or one short allele and one medium allele indicates that the potential donor would provide MSCs that are superior for therapeutic uses; (b) selecting those donors having such MSCs; (c) obtaining (or providing) a population of human MSCs; and (d) administering an effective dose of the therapeutically effective MSCs to the subject, thereby treating the MSC-related pathology in the subject.

Also provided are populations of therapeutically effective MSCs for use in treating an MSC-related pathology, wherein the population of therapeutically effective MSCs is obtained by: (a) analyzing the number of GT repeats present in the HO-1 promoter region of both alleles of a potential human donor to determine whether the potential donor has short, medium, or long alleles, wherein the presence of two short alleles, two medium alleles, or one short allele and one medium allele indicates that the potential donor would provide MSCs that are superior for therapeutic uses; and (b) selecting those donors having such MSCs.

The MSCs can be administered to the patient using any route of administration known in the art. By way of non-limiting example, the MSCs can be administered intra-arterially or intravenously to the patient. In some embodiments, the MSCs are administered to the patient in a biologically and physiologically compatible solution. Preferably, the solution is not enriched for pluripotent hematopoietic stem cells.

In some embodiments of the invention, an effective amount of MSCs is between about 7×10⁵ and about 7×10⁶ cells/kg.

The donor can be autologous or allogeneic to the subject. The number of GT repeats can be analyzed from a blood sample, an MSC cryopreserved sample, a sample from a MCB, a bone marrow sample, and/or any other suitable genetic material. Those skilled in the art will recognize that it is possible to obtain or provide MSCs from an ex vivo source/sample. Moreover, the treated subject may be any living organisms such as humans (non-human animals (e.g., monkeys, cows, sheep, horses, pigs, cattle, goats, dogs, cats, mice, or rats), cultured cells therefrom, and transgenic species thereof.

The MSC-related pathology to be treated may be one or more of the following: a neurological pathology (e.g., stroke), an inflammatory pathology (e.g., multi-organ failure), a renal pathology (e.g., acute kidney injury, acute renal failure, chronic renal failure, chronic kidney disease, transplant, diabetic nephrology, and hypertensive nephrology), a hepatic pathology, a cardiovascular pathology, a retinal pathology, a muscular pathology, a bone-related pathology, a gastrointestinal pathology, a skin related pathology and/or a metabolic pathology (e.g., diabetes).

Moreover, in these methods, a short allele has ≦26 (e.g., between about 21 and about 26) GT repeats, a medium allele has between about 27 and about 32 GT repeats, and a long allele has >32 (e.g., between about 33 and about 44) GT repeats.

The invention also provides kits containing (in one or more containers) reagents for the analyzing the number of GT repeats present in the HO-1 promoter region of both alleles in a population of human MSCs. Such kits may also include instructions for use. Such kits may also additionally contain reagents (in one or more containers) for culturing human MSCs and/or reagents for freezing human MSCs. In various embodiments, the reagents for analyzing the number of GT repeats contain reagents for use in DNA Fragment Length Analysis and/or reagents for use with polymerase chain reaction (PCR).

The invention further provides methods of producing dosage forms of therapeutically effective human MSCs.

In one embodiment, such methods involve the steps of (a) obtaining (or providing) a population of human MSCs; (b) analyzing the number of GT repeats present in the HO-1 promoter region of both alleles to determine whether the MSCs have short (i.e., ≦26 GT repeats or between about 21 and about 26 GT repeats), medium (i.e., between about 27 and about 32 GT repeats), or long (i.e., >32 GT repeats or between about 33 and about 44 GT repeats) alleles, wherein the presence of two short alleles, two medium alleles, or one short allele and one medium allele indicates that the population contains MSCs that are therapeutically effective; and (c) selecting therapeutically effective human MSCs, thereby producing a dosage form of human MSCs.

In another embodiment, these methods involve the steps of (a) analyzing the number of GT repeats present in the HO-1 promoter region of both alleles in genetic material from a potential human donor to determine whether the potential donor has short (i.e., ≦26 GT repeats or between about 21 and about 26 GT repeats), medium (i.e., between about 27 and about 32 GT repeats), or long (i.e., >32 GT repeats or between about 33 and about 44 GT repeats) alleles, wherein the presence of two short alleles, two medium alleles, or one short allele and one medium allele indicates that the potential donor would provide MSCs that are superior for therapeutic uses, and (b) selecting those donors having such MSCs, thereby producing a therapeutically effective dosage form of human MSCs.

Preferably, in any of the methods of producing a dosage form disclosed herein, analyzing the number of GT repeats in potential bone marrow donors is done prior to bone marrow donation and/or isolation of MSCs. However, those skilled in the art will recognize that analyzing the number of GT repeats present in the HO-1 promoter region of both alleles can also be done on established populations of MSCs and/or on existing MCBs.

In any of the methods of producing a dosage form described herein, therapeutically effective MSCs contain two short alleles, two medium alleles, or one medium and one short allele. Moreover, MSCs containing one or more long alleles are less therapeutically effective, and, thus, ideally, will be excluded from any of the methods and compositions described herein.

The MSCs in the dosage form may be autologous or allogeneic to the subject. Moreover, the subject may be any living organism such as humans, non-human animals (e.g., monkeys, cows, sheep, horses, pigs, cattle, goats, dogs, cats, mice, rats), cultured cells therefrom, and transgenic species thereof.

Human MSCs in the dosage forms may be obtained (or provided) from any suitable source known in the art, for example, a cryopreserved sample, a sample from a MCB, a bone marrow sample, an adipose tissue sample, and/or a cord blood sample. Likewise, the number of GT repeats present in the HO-1 promoter region of both alleles of a potential human donor can be analyzed from a blood sample, a cryopreserved MSC sample, a sample from a MCB, a bone marrow sample, and/or any other suitable genetic material. Those skilled in the art will recognize that it is possible to obtain or provide MSCs from an ex vivo source/sample.

Also provided are populations of human MSCs, wherein the human MSCs in the population preferably have 32 or fewer GT repeats in both alleles of the HO-1 promoter region (e.g., between 21 and 32). For example, the human MSCs in the population contain two short alleles, two medium alleles, or one short allele and one medium allele of the HO-1 promoter region. Therefore, ideally, the population of human MSCs does not contain any long alleles.

In any of the populations described herein, the population of human MSCs has been cultured in PL supplemented culture media. Those skilled in the art will recognize that MSCs that have been cultured in PL supplemented culture media will express Prickle 1 to a higher degree than MSCs that have been cultured in fetal bovine serum (FBS) supplemented culture media. For example, the population of human MSCs expresses Prickle 1 to an eight-fold higher degree than MSCs that have been cultured in FBS supplemented culture media. (See, e.g., Lange et al., Cellular Therapy and Transplantation 1:49-53 (2008), which is herein incorporated by reference in its entirety). Those skilled in the art will recognize that a population of human MSCs that has been cultured in PL may be less immunogenic than MSCs that have been cultured in FBS supplemented culture media. Moreover, the use of PL instead of FBS supplemented culture media reduces infectious risk and overall safety and regulatory concerns associated with the use of FBS.

In still a further embodiment, the invention provides populations of human MSCs that contain at least 75% human MSCs (e.g., at least 80%, at least 85%, at least 90%, at least 95%, at least 96%, at least 97%, at least 98%, at least 99%, or 100%), wherein: a) the human MSCs in the population contain between 21 and 32 GT repeats in each allele of the HO-1 promoter region; b) the human MSCs in the population have been cultured in PL supplemented culture media and express Prickle 1 at a higher degree than MSCs that have been cultured in FBS supplemented culture media; c) the human MSCs are cultured to between 75 and 100%% (e.g., between 75 and 95%, between 75 and 90%, between 75 and 85%, between 75 and 80%, between 80 and 100%, between 80 and 95%, between 80 and 90%, between 80 and 85%, between 85 and 100%, between 85 and 95%, between 85 and 90%, between 90 and 100%, between 90 and 95%, and/or between 95-100%) confluence; d) the population does not contain detectable levels of infectious agents; and e) the human MSCs in the population have only undergone fewer than 30 population doublings. Preferably, in some embodiments, at least 70% of the cells in the population (e.g., at least 75%, at least 80%, at least 85%, at least 90%, at least 95%, at least 96%, at least 97%, at least 98%, at least 99%, or 100%) remain viable following expansion in culture. Moreover, the human MSCs in the population can be cultured in an open system or a closed system (e.g., a bioreactor or a cell factory). These cells can be aliquoted into individual vials.

In some embodiments, the MSCs are genetically modified, wherein renoprotective potency of said cells is augmented by genetic modification prior to administration to the patient.

In any of the methods described herein, the MSCs can be pre-differentiated in vitro prior to administration to the patient. By way of non-limiting example, the MSCs are pre-differentiated into endothelial cells and/or into renal tubular cells.

Finally, the invention further provides methods for treating pathology in a subject by administering a therapeutically effective amount of any of the human MSC populations of the invention to the subject. In such methods, the population of human MSCs may be either autologous or allogeneic to the subject.

Also provided are populations of human MSCs for use in treating pathology in a subject, wherein the population of human MSCs is for administration to the subject in a therapeutically effective amount.

For example, the pathology to be treated in accordance with these methods may be neurological pathology (e.g., stroke), an inflammatory pathology (e.g., multi-organ failure), a renal pathology (e.g., acute kidney injury, acute renal failure, chronic renal failure, chronic kidney disease, transplant, diabetic nephrology, and hypertensive nephrology), a hepatic pathology, a cardiovascular pathology, a retinal pathology, a muscular pathology, a bone-related pathology, a gastrointestinal pathology, a skin related pathology and/or a metabolic pathology (e.g., diabetes).

### BRIEF DESCRIPTION OF THE DRAWINGS

FIG. 1 is a table showing the result of HO-1 GT repeat length analysis. FIG. 1A shows the PCR Fragment size, GT repeat number, and GT repeat length of both alleles for 25 different MCB samples. FIG. 1B is a conversion table showing the correlation between PCR Fragment size (in base pairs), HO-1 GT repeat number, and HO-1 GT repeat length.

FIG. 2 is a table showing the result of HO-1 GT repeat length analysis for peripheral samples obtained from potential bone marrow donors or potential volunteers being evaluated for future development of additional MSCs. FIG. 2A shows the PCR Fragment size, GT repeat number, and GT repeat length of two alleles for samples from 53 potential donors or research volunteers. FIG. 2B is a conversion table showing the correlation between PCR Fragment size (in base pairs), HO-1 GT repeat number, and HO-1 GT repeat length.

### DETAILED DESCRIPTION OF THE INVENTION

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural references unless the context clearly dictates otherwise. All patents and publications cited in this specification are incorporated by reference in their entirety.

For convenience, certain terms used in the specification, examples and claims are collected here. Unless otherwise defined, all technical and scientific terms used herein have the same meaning as commonly understood by one of ordinary skill in the art to which this invention pertains.

Hemeoxygenases are the enzymes responsible for the catalysis of heme molecules in mammalian cells. They play a vital biological role in the response to cellular injury, which can result in the denaturing of heme-containing proteins with subsequent release of heme moiety. If not promptly metabolized, free heme molecules can act as a potent source of secondary oxidative stress. (See Ferenbach et al., Nephron Exp Nephrol 115:e33-e37 (2010), which is herein incorporated by reference in its entirety).

The heme oxygenase system catalyzes the rate-limiting step in heme degradation, namely the production of equimolar quantities of biliverdin, iron, and carbon monoxide (CO). (See Sikorski et al., Am J. Physiol Renal Physiol 286:F425-441 (2004), which is incorporated herein by reference). Constitutively expressed hemoxygenase-2 and -3 contribute a basal level of heme metabolism. However, heme degradation occurs primarily through an inducible enzyme, hemoxygenase-1 (HO-1).

Because heme molecules are contained all nucleated cells in the body, including those in the kidney, there is also a need for widespread availability of HO-1 for heme metabolism.

Moreover, because HO-1 has been shown to play a cytoprotective role, it may be a target for therapeutic intervention. The protective effects of HO-1 are mediated through one or more of several potential mechanisms. (See Sikorski et al. at page F426). In addition, the reaction products of heme metabolism possess important antioxidant, anti-inflammatory, and anti-apoptotic functions. In fact, those skilled in the art will recognize that two of the products of heme metabolism (biliverdin and CO) possess immunomodulatory, anti-apoptotic and, for CO, vasoactive properties. (See Ferenbach et al, at page e34). However, it is also possible that HO-1 plays a dual role in tissue pathology—it may not be therapeutic in all instances, as each of the products of the reaction can be potentially injurious as well. (See Sikorski et al. at page F426).

The present invention provides a method for assaying and/or evaluating donors of MSCs and/or purified MSCs for their therapeutic effectiveness or potency. The invention is based upon the finding that the number of GT repeats in the human HO-1 promoter region of MSCs may be indicative of the therapeutic efficacy of the MSCs. Analyzing the number of GT repeats in both donor alleles (whether obtained or provided from a cryopreserved MSC sample, from fresh blood, from a MCB and/or from any other suitable genetic material), helps to determine whether the MSC population is enriched to be robust, and, thus, be therapeutically effective.

Preferably, the number of GT repeats in both HO-1 alleles is not too long. Indeed, as described herein, MSCs having fewer GT repeats in both HO-1 alleles express higher HO-1 protein levels are more likely to be therapeutically effective.

A (GT)n repeat region that can function as a negative regulatory region is located between −190 and −270 of the human HO-1 promoter and is absent in the mouse HO-1 gene. (See Sikorski et al. at page F429). In addition, length polymorphisms of this region vary between subjects and correlate with activity of various diseases, such as emphysema, coronary artery disease, and other disorders. Typically, individuals with shorter repeats (<25) demonstrate higher induced HO-1 protein levels and milder disease, whereas individuals with longer repeats have lower HO-1 levels and more severe disease. (See Sikorski et al., Am J. Physiol. Renal Physiol. 286:F424-F441 (2004); Zarjou et al., Am J Physiol Renal Physiol 300:F254-F262 (2011); Exner et al., Free Radical Biology & Medicine 37(8):1097-104 (2004), which are herein incorporated by reference in their entireties).

As used herein, “patient,” “individual,” “subject” or “host” refers to either a human or a non-human animal.

As used herein, the term “short allele” refers to MSC HO-1 alleles having ≦26 GT repeats in the human HO-1 promoter region.

As used herein, the term “medium allele” refers to MSC HO-1 alleles having between 27 and 32 GT repeats in the human HO-1 promoter region (i.e., 27, 28, 29, 30, 31, and 32).

As used herein, the term “long allele” refers to MSC HO-1 alleles having >32 GT repeats in the human HO-1 promoter region.

Studies in mice have demonstrated that HO-1 is essential for their therapeutic potential in cisplatin-induced AKI. (See Zarjou et al., Am J Physiol Renal Physiol 300:F254-F262 (2011)). Moreover, the absence of HO-1 expression in MSCs limit their protective paracrine effects including the angiogenic potential of MSCs and for growth factor and/or reparative factor expression and secretion by MSCs. (See Zarjou et al. at p. F260).

Moreover, the number of GT repeats in the HO-1 promoter region of any nucleated cell of the human body may be measured by any method known in the art. For example, DNA Fragment Length Analysis can be used. Briefly, PCR is used to amplify fragments from both HO-1 alleles within cells using PCR primers that flank the HO-1 promoter region containing the GT repeats. The resulting PCR fragments are separated on a column and the “predicted” sizes are reported (in base pairs).

DNA Fragment Length Analysis is, thus, able to report relative size differences between different alleles. The absolute size of the PCR fragments can subsequently be determined using methods well known to those of ordinary skill in the relevant art.

Two clinical-grade MSC populations were each designated as a “Master Cell Bank” or MCB. These MCBs are MCB 808 and MCB 810 and were used in establishing the method of genotyping by fragment length analysis.

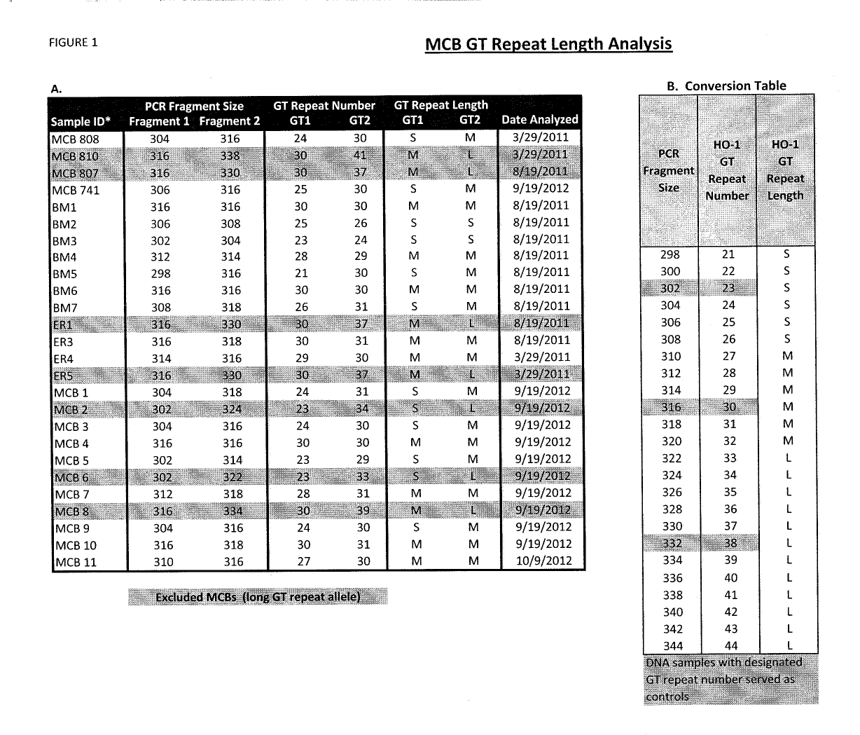

FIG. 1 is a spreadsheet showing completed genotyping data for 25 MSC, including the MCBs 808 and 810. Only MCBs without any long alleles will be used for future manufacturing of MSC doses suitable for therapeutic use.

DNA Fragment Length Analysis (see Exner et al., Free Radical Biology & Medicine 37(8):1097-104 (2004)) is used to determine the number of GT repeats. Briefly, PCR is used to amplify fragments from both HO-1 alleles per MSC using PCR primers, one of which is fluorescently labeled (for example, with FAM), that flank the HO-1 promoter region containing the GT repeats. The resulting PCR fragments are separated on a column (for example, at an external vendor), and the “predicted” DNA fragment sizes are reported (in base pairs).

DNA Fragment Length Analysis is a commonly used method for determining the length of FAM-labeled PCR fragments. However, fragment length analysis only predicts the relative size of different fragments and the relative differences between different alleles. Based upon the fragment length data, it is believed that a PCR fragment size of 302 base pairs corresponds to 23 GT repeats. However, those skilled in the art will appreciate that the apparent fragment length could differ on a different column.

Fragment length was confirmed by synthesizing control DNA fragments with pre-specified GT repeat lengths that were cloned into a plasmid vector. Specifically, three different “known” DNA fragments were synthesized with a specified number of GT repeats (23, 30, or 38). As a control, the PCR and Fragment Length Analysis were performed on each of these three DNA vectors (representing a short, medium, and long allele). These control studies confirmed that the initial data was accurate with respect to the GT repeat number. In particular, these control studies confirmed that a PCR fragment size of 302 base pairs corresponds to 23 GT repeats.

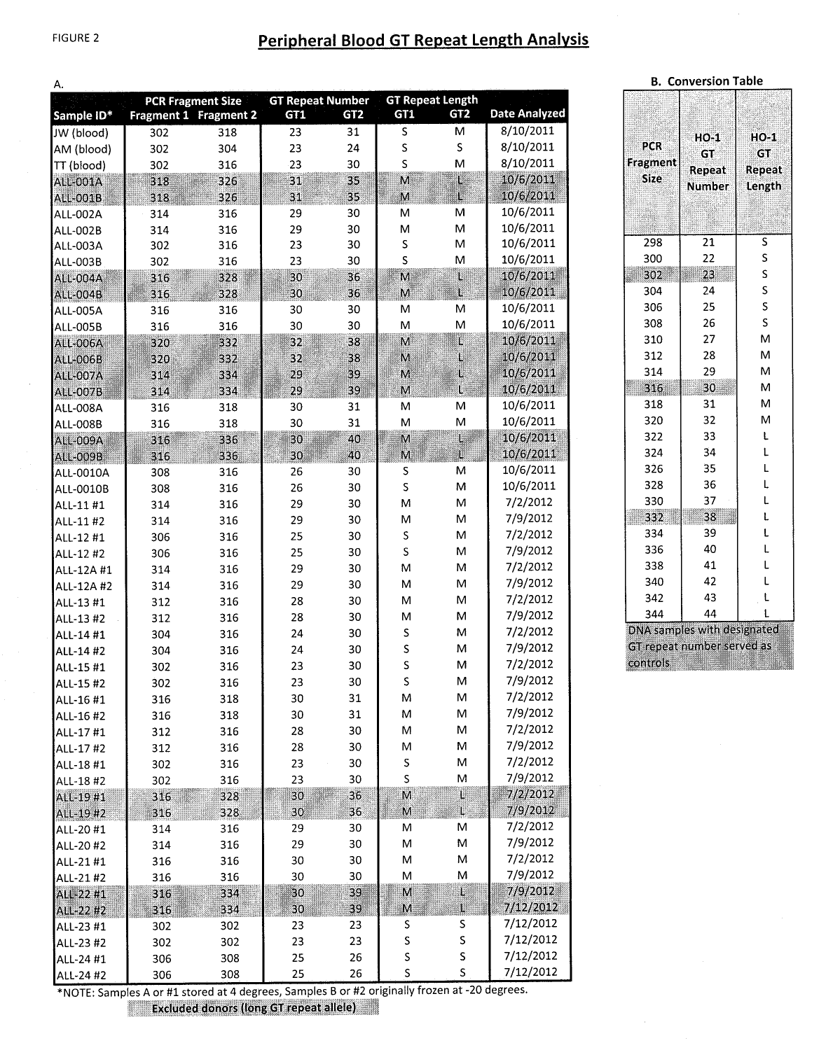

In addition, the data for these 3 control DNA vectors does not change the relative difference of the GT repeat numbers between different alleles. For example, MCB 810 has one allele with 11 more GT repeats than its other allele (41 and 30, respectively). FIG. 2A shows the results of GT repeat analysis performed on data from 50 samples obtained from potential bone marrow donors that are being evaluated for future development of additional MSCs (i.e., to determine their potential therapeutic effectiveness), as well as samples from three research volunteers. FIG. 2B shows the Conversion Table used to determine whether the HO-1 alleles from each donor or research volunteer are classified as short, medium, or long.

In accordance with the methods of the instant invention, donors or MSCs will be excluded if they have one or more long GT repeat alleles. Thus, only those donors or MSCs having two short alleles, two medium alleles, or one medium and one short allele will be accepted. Only MSCs without a long allele will be used clinically.

In other embodiments, HO-1 protein expression levels in MSCs can be induced, for example, by using cobalt protoporphyrin (CoPP) or hypoxia. The MSCs to be studied include, for example, BM3 (S, S), ER4 (M, M), ER5 (M, L), and 810 (M, L). (See FIG. 1).

According to certain embodiments of the invention, other MSC markers are also measured. For example, the presence of CD105 and/or CD90 is measured in some embodiments. In other embodiments, the absence of CD34 and/or CD45 is measured. The presence of CD105 and/or CD90 as well as the absence of CD34 and/or CD45 is indicative of the MSC phenotype. In other embodiments, adipogenic, osteogenic and/or chondrogenic assays are used to show that the MSCs possess the characteristic ability of trilineage differentiation.

### Mesenchymal Stem Cells Cultured in Platelet Lysate (PL) Supplemented Media

MSCs may be passaged or expanded according to any methods known in the art. For example, published PCT application WO2010/017216, which is incorporated herein by reference in its entirety, describes methods for the culture and expansion of MSCs in PL supplemented media.

The invention provides MSCs with unique properties that make them particularly beneficial for use in the treatment of neurological or kidney pathology. The MSCs of the invention are grown in media containing PL, as described in greater detail below. The culturing of MSCs in PL-supplemented media creates MSCs that are more protective against ischemia-reperfusion damage than MSCs grown in FBS.

The MSCs of the invention, cultured in PL-supplemented media constitute a population with (i) surface expression of the antigens CD105, CD90, CD73 and MHC I, but lacking hematopoietic markers CD45, CD34 and CD14; (ii) preservation of the multipotent trilineage (osteoblasts, adipocytes and chondrocytes) differentiation capability after expansion with PL, however the adipogenic differentiation was delayed and needed longer times of induction. This decreased adipogenic/lipogenic ability is a favorable property because in mice the intra-arterial injection of MSCs for treatment of chronic kidney injury has revealed formation of adipocytes (Kunter et al., J Am Soc Nephrol 2007 June; 18(6):1754-64). These results are reflected in the gene expression profile of PL-generated cells revealing a down-regulation of genes involved in fatty acid metabolism, described in greater detail below.

The MSCs of the invention, cultured in PL-supplemented media have been described to act immunomodulatory by impairing T-cell activation without inducing anergy. There is a dilution of this effect in vitro in mixed lymphocyte cultures (MLC) leading eventually to an activation of T-cells if decreasing amounts of MSCs, not cultured in PL-supplemented media, are added to the MLC reaction. This activation process is not observed when PL-generated MSCs are used in the MLC as third party, as shown in greater detail below. It was concluded that the MSCs of the invention, cultured in PL-supplemented media are less immunogenic and that growing MSCs in FCSFBS-supplemented media may act as a strong antigen or at least has adjuvant function in T-cell stimulation. This result again is reflected in differential gene expression showing a down-regulation of MHC II molecules verifying the decreased immunostimulation by MSC, as shown below.

Moreover, the MSCs of the invention, cultured in PL-supplemented media show up-regulation of genes involved in the cell cycle (e.g. cyclins and cyclin dependent kinases) and the DNA replication and purine metabolism when compared to MSCs cultured in FBS-supplemented media. On the other hand, genes functionally active in cell adhesion/extracellular matrix (ECM)-receptor interaction, differentiation/development, TGF-β signaling and TSP-I induced apoptosis could be shown to be down-regulated in the MSCs of the invention, cultured in PL-supplemented media when compared to MSCs cultured in FBS-supplemented media, again supporting the results of faster growth and accelerated expansion.

The MSCs of the invention, cultured in PL-supplemented media when intra-arterially administered lead to improvement of repair and regeneration of injured tissue by ameliorating local inflammation, decreasing apoptosis, and by delivering growth factors and other mediators needed for the repair and/or regeneration of the damaged cells. Injured cells (for example, in the kidney) secrete SDF-1 that homes MSCs carrying the chemokine receptor 4 (CXCR4) to the site of injury.

The MSCs of the invention, cultured in PL-supplemented media are particularly good candidates for regenerative therapy in central nervous system (CNS) damage. They express the gene Prickle 1 to an eight-fold higher degree compared to MSCs cultured in FBS-supplemented media which is involved in neuroregeneration. Mouse Prickle 1 and Prickle 2 are expressed in postmitotic neurons and promote neuronal outgrowth (Okuda et al., FEBS Lett. 2007 Oct. 2; 581(24):4754-60). These differentially regulated genes would favor the use of PL cultured hMSC for regeneration of neuronal injury.

Additionally, the expression of retinoic acid receptor (RAR) responsive gene TIG1, shows 12 fold higher expression in the MSCs of the invention, cultured in PL-supplemented media) (Liang et al. Nature Genetics 2007; 39(2):178-188), Keratin 18 (9 fold higher expression in the MSCs of the invention, cultured in PL-supplemented media) (Bühler et al, Mol Cancer Res. 2005; 3(7):365-71), CRBP1 (cellular retinol binding protein 1, 5.7 fold higher expression in the MSCs of the invention cultured in PL-supplemented media) (Roberts et al., DNA Cell Biol. 2002; 21(1):11-9.) and Prickle 1 suggest a less tumorigenic phenotype of the MSCs of the invention, cultured in PL-supplemented media.

Furthermore, MSCs grown in PL-supplemented medium are more protective against ischemia-reperfusion damage than MSCs grown in FBS-supplemented medium.

### Methods of Producing Mesenchymal Stem Cells

In certain embodiments, the MSCs of the invention are cultured in media supplemented with PL. In one embodiment of the method of producing MSCs of the invention, the starting material for the MSCs is bone marrow isolated from healthy donors. Preferably, these donors are mammals. More preferably, these mammals are humans. In one embodiment of the method of producing MSCs of the invention, the bone marrow is cultured in tissue culture cell factories between 1 and 10 days (i.e., 1, 2, 3, 4, 5, 6, 7, 8, 9, or 10 days) prior to washing non-adherent cells from the cell factory. Optionally, the number of days of culture of bone marrow cells prior to washing non-adherent cells is 2 to 4 (i.e., 2, 3 or 4) days. Preferably the bone marrow is cultured in PL containing media. 100-120 mL (i.e., 100, 101, 102, 103, 104, 105, 106, 107, 108, 109, 110, 111, 112, 113, 114, 115, 116, 117, 118, 119, or 120 mL) of bone marrow aspirate is cultured in between 1,000 to 2,000 mL (i.e., 1,000; 1,100; 1,200; 1,300; 1,400; 1,500; 1,600; 1,700; 1,800; 1,900; or 2,000 mL) of PL supplemented media (or enough media for optimal cell growth in a given culture vessel) in multi-layered cell factory or other adequate tissue culture vessels.

After washing away the non-adherent cells, the adherent cells are also cultured in media that has been supplemented with PL. Thrombocytes (platelets) are a well-characterized human product already widely used clinically for patients in need. Platelets are known to produce a wide variety of factors, e.g. PDGF-BB, TGF-β, IGF-1, and VEGF. In one embodiment of the method of producing MSCs of the invention, an optimized preparation of PL is used. This optimized preparation of PL is made up of pooled platelet rich plasma (PRP) from at least 5 to 20 donors (i.e., 5, 6, 7, 8, 9, 10, 11, 12, 13, 14, 15, 16, 17, 18, 19, 20, or more) with a minimal concentration of 2×10⁹ to 4×10⁹ thrombocytes/mL (i.e., 2×10⁹, 2.5×10⁹, 3×10⁹, 3.5×10⁹, or 4×10⁹ thrombocytes/mL).

According to preferred embodiments of the method of producing MSCs of the invention, PL was prepared either from pooled thrombocyte concentrates designed for human use or from 7-13 (i.e., 7, 8, 9, 10, 11, 12, or 13) pooled buffy coats after centrifugation with 200×g for 20 min. Preferably, the PRP was aliquoted into small portions, frozen at −80° C., and thawed immediately before use. Thawing of PRP causes lysis of thrombocytes, generating PL, and release of growth factors that facilitate robust MSC growth. PL-containing medium was prepared freshly for each lot production. In a preferred embodiment, medium contained αMEM (minimum essential medium alpha) as basic medium supplemented with 1 to 5 IU Heparin/mL (i.e., 1 IU Heparin/mL, 1.5 IU Heparin/mL, 2 IU Heparin/mL, 2.5 IU Heparin/mL, 3 IU Heparin/mL, 3.5 IU Heparin/mL, 4 IU Heparin/mL, 4.5 IU Heparin/mL, or 5 IU Heparin/mL) medium (source: Ratiopharm) and 5% of freshly thawed PL, which can be used for up to 30 days without significant loss of MSC growth supporting properties. The method of producing MSCs of the invention, uses a method to prepare PL that differs from others according to the thrombocyte concentration and centrifugation forces. The composition of this PL is described in greater detail, below.

In one embodiment of the method of producing MSCs, the adherent cells are cultured in PL-supplemented media at 37° C. with approximately 5% CO₂ under hypoxic conditions. Preferably, the hypoxic conditions are an atmosphere of 5% O₂. In some situations hypoxic culture conditions allow MSCs to grow more quickly. This allows for a reduction of days needed to grow the cells to approximately 80-100% confluence. Generally, it reduces the growing time by three days. In another embodiment of the method of producing MSCs of the invention, the adherent cells are cultured in PL-supplemented media at 37° C. with approximately 5% CO₂ under normoxic conditions, i.e. wherein the O₂ concentration is the same as atmospheric O₂, approximately 20.9%. Preferably, the adherent cells are cultured between 2 and 10 days (i.e., 2, 3, 4, 5, 6, 7, 8, 9, or 10 days), being fed every 3-8 (e.g., 3-7, 3-6, 3-5, 3-4, 4-8, 4-7, 4-6, 4-5, 5-8, 5-7, 5-6, 6-8, or 6-7) days with PL-supplemented media. In one embodiment of the method of producing MSCs of the invention, the adherent cells are grown to between approximately 80 and 100% confluence. Preferably, once this level of confluence is reached, the cell monolayers are detached from the culture vessel enzymatically by using recombinant trypsin. The detached cells in suspension are plated for subsequent culture. The process of successive detaching and plating of cells is called passage.

In certain embodiments, the population of cells that is isolated from the culture vessel is between 50-99% MSCs. In other embodiments, isolated MSCs are enriched in MSCs so that 25%, 30%, 35%, 40%, 45%, 50%, 55%, 60%, 65%, 70%, 75%, 80%, 85%, 90%, 95%, 96%, 97%, 98% or 99% of the cell population are MSCs. In other embodiments, the MSCs are greater than 95% of the isolated cell population.

Preferably, the MSCs used in any of the methods, compositions, and kits described herein are free of infectious agents. In some embodiments, the MSCs have undergone fewer than 30 population doublings and are cultured to approximately 80 to 100% confluence. Moreover, using the various methods described herein, MSC cell viability should be greater or equal to 70%.

In another embodiment of the method of producing MSCs of the invention, the cells are frozen after they are released from the tissue culture vessel. Freezing is performed in a step-wise manner in a physiologically acceptable carrier, 5 to 10% (i.e., 5, 6, 7, 8, 9, or 10%) human serum albumin (HSA) and 5 to 10% (i.e., 5, 6, 7, 8, 9, or 10%) DMSO. Thawing is also performed in a step-wise manner. Preferably, when thawed, the frozen MSCs of the invention are diluted 4:1 to reduce DMSO concentration. In this case, frozen MSCs of the invention are thawed quickly at 37° C. and administered intravenously without any dilution or washings. Optionally the cells are administered following any protocol that is adequate for the transplantation of hematopoietic stem cells (HSCs). Preferably, the serum albumin is HSA.

In another embodiment of the method of producing MSCs of the invention, the cells are frozen in aliquots of 10⁴-10¹² (i.e., 10⁴, 10⁵, 10⁶, 10⁷, 10⁸, 10⁹, 10¹⁰, 10¹¹, or 10¹²) cells in 10 to 20 mL of physiologically acceptable carrier and HSA in a presence of cryoprotectant (5-10% (i.e., 5, 6, 7, 8, 9, or 10%) DMSO). In another embodiment of the method of producing MSCs of the invention, the cells are frozen in aliquots of 10⁶-10⁸ (i.e., 1×10⁶, 5×10⁶, 1×10⁷, 5×10⁷, 1×10⁸, or 5×10⁸) cells in 10 to 20 mL (i.e., 10, 11, 12, 13, 14, 15, 16, 17, 18, 19, or 20 mL) of physiologically acceptable carrier and HSA. In another embodiment of the method of producing MSCs of the invention, the cells administered in a dose of 10⁶-10⁸ (i.e., 1×10⁶, 5×10⁶, 1×10⁷, 5×10⁷, 1×10⁸, or 5×10⁸) cells per kg of subject body weight, in 50-100 mL (i.e., 50, 55, 60, 65, 70, 75, 80, 85, 90, 95, or 100 mL) of physiologically acceptable carrier and HSA. In one aspect of these embodiments, when a therapeutic dose is being assembled, the appropriate number of cryovials is thawed in order to thaw the appropriate number of cells for the therapeutic dose based on the patient's body weight. Preferably, after DMSO is diluted, the number of cryovials chosen is placed in a sterile infusion bag with 5-10% (i.e., 5, 6, 7, 8, 9, or 10%) HSA. Once in the bag, the MSCs do not aggregate and viability remains greater than 70% (i.e., greater than 70%, greater than 75%, greater than 80%, greater than 85%, greater than 90%, greater than 95%, greater than 96%, greater than 97%, greater than 98%, greater than 99%, or 100%) even when the MSCs are stored at room temperature for at least 8 to 10 hours. This provides ample time to administer the MSCs of the invention to a patient in an operating room. Optionally, the physiologically acceptable carrier is PlasmaLyte A. Preferably the HSA is present at a concentration of 5% w/v. Suspending the 10⁶-10⁸ (i.e., 1×10⁶, 5×10⁶, 1×10⁷, 5×10⁷, 1×10⁸, or 5×10⁸) MSCs of the invention in greater than 50 mL (i.e., greater than 50, greater than 55, greater than 60, greater than 65, greater than 70, greater than 75, greater than 80, greater than 85, greater than 90, greater than 95, greater than 100, greater than 110, greater than 120, greater than 130, greater than 140, greater than 150, greater than 160, greater than 170, greater than 180, greater than 200, greater than 225, greater than 250, greater than 275, greater than 300, greater than 325, greater than 350, greater than 375, greater than 400, greater than 425, greater than 450, greater than 475, greater than 500, or more) of physiological carrier is critical to their biological activity. If the cells are suspended in lower volumes, the cells are prone to aggregation. Administration of aggregated MSCs to mammalian subjects has resulted in cardiac infarction. Thus, it is crucial that non-aggregated MSCs be administered according to the methods of the invention. The presence of HSA is also critical because it prevents aggregation of the MSCs and also prevents the cells from sticking to plastic containers the cells pass through when administered to subjects.

In certain embodiments of the method of producing MSCs of the invention, the culture system is used in conjunction with a medium for expansion of MSCs which does not contain any animal proteins, e.g. PL. FBS has been connected with adverse effects after in vivo application of FBS-expanded cells, e.g. formation of anti-FBS antibodies, anaphylactic or Arthus-like immune reactions or arrhythmias after cellular cardioplasty. FBS may introduce unwanted animal xenogeneic antigens, viral, prion and zoonose contaminations into cell preparations making new alternatives desirable.

### Manufacturing Summary

In one embodiment, a bone marrow aspirate is suspended in culture media and then plated in multilayer cell factory in a closed system manner. Mesenchymal progenitors naturally attach to the surface of the cell factory and then expand after several passages to become a relatively homogeneous population of MSC. After 1 to 4 days the cells remaining in suspension are washed out of the cell factory and discarded.

When the MSCs have expanded to cover the culture surface, they are enzymatically detached and harvested. The harvested cells are seeded in more cell factories and the expansion process is repeated. Feeding and harvesting of the cells takes place in a completely closed system using sterile welders.

After 2 expansion rounds (10-20 days) the MCB cells are harvested and cryopreserved in vapor phase liquid N2 at <−130° C. Representative MCB units are tested for sterility, mycoplasma, endotoxin, identity by flow cytometry and trilineage differentiation, as well as an array of viral tests.

Preferably, bone marrow aspirates are donated by healthy adult volunteers. Potential donors undergo rigorous testing including health questionnaire, physical examination, and testing for various infectious diseases. A summary of pre-testing of donors is given below:

| Assay/Agent/Disease | Test | Specification |
|---|---|---|
| Complete Blood Count | CBC with differential and platelet | Approved by medical |
| | count | director |
| Comprehensive Metabolic Profile | CMP 14 | Approved by medical |
| | | director |
| ABO Rh Blood Group and Type | ABO Rh | For information only |
| HLA Typing | A, B, C, DR Beta I | For information only |
| Human Immunodeficiency Viruses 1 | Antibody HIV-1, HIV-2 | Non reactive (NR) |
| and 2 (HIV)* | | |
| HIV-1 | HIV-1 nucleic acid test (NAT) | Negative or NR |
| Hepatitis B Virus (HBV)* | Hep B surface Antigen (HBsAg) | Non reactive |
| Hepatitis B Virus | Antibody Hep B core (total) | Non reactive |
| Hepatitis B Virus | HBV NAT | Negative or NR |
| Hepatitis C Virus (HCV)* | Antibody HCV | Non reactive |
| Hepatitis C Virus | HCV NAT | Negative or NR |
| West Nile Virus (WNV) | WNV NAT | Negative or NR |
| Syphilis (Treponema pallidum)* | RPR (rapid plasma reagin) | Non reactive/Reactive |
| | (nonspecific test) | |
| Syphilis (Treponema pallidum) | FTA-ABS (performed if RPR is | Non reactive |
| | reactive) (Fluorescent Treponema | |
| | Antibody ABSorbed) | |
| Human T cell Lymphotropic Virus | Antibody HTLV I/II | Non reactive |
| Types I & II* | | |
| Cytomegalovirus (CMV)* | Antibody CMV Total | Non reactive |
| Epstein-Barr Virus (EBV) | EBV Viral Capsid Antigen (VCA) | Non reactive |
| | IgM | |
| Human transmissible spongiform | Health questionnaire | No exposure |
| Encephalopathy (TSE), including | | |
| Creutzfeldt-Jakob disease (CJD)* | | |
| Communicable disease risks | Health questionnaire | No exposure |
| associated with xenotransplantation* | | |

A summary of post donation testing of cells (e.g., MCB testing) is given below:

| | | Limits or Range |
|---|---|---|
| Test and Specifications | Test Procedure | Number |
| Endotoxin | LAL | <5 EU/100 ml |
| Sterility | USP <71> | Negative |
| Mycoplasma | FDA PTC | Negative |
| Cell Yield per Vial | Nucleocounter | ≧7 × 10⁶ |
| (Post Cryopreservation) | | |
| Test Code 6347 | | |
| Cell Viability (Post Cryopreservation) | Nucleocounter | ≧70% |

FACS Analysis

| | | |
|---|---|---|
| CD34 | FACS | ≦10% |
| CD45 | | ≦10% |
| CD90 | | ≧90% |
| CD44 | | ≧90% |
| CD73 | | ≧90% |
| CD105 | | ≧90% |
| HLA-DR | | ≦10% |

Trilineage Analysis

| | |
|---|---|
| Osteogenic differentiation | Positive |
| Adipogenic differentiation | Positive |
| Chondrogenic differentiation | Positive |

| Assay/Agent/Disease | Test | Specification |
|---|---|---|
| Assay for the Detection of HIV I/II | PCR | Not Detected |
| Assay for the Detection of HBV | PCR | Not Detected |
| Assay for the Detection of HCV | PCR | Not Detected |
| Assay for the Detection of EBV | PCR | Not Detected |
| Assay for the Detection of CMV | PCR | Not Detected |
| Assay for the Detection of HHV-6 | PCR | Not Detected |
| Assay for the Detection of HHV-7 | PCR | Not Detected |
| Assay for the Detection of HHV-8 | PCR | Not Detected |
| Assay for the Detection of HTLV I/II | PCR | Not Detected |
| Assay for the Detection of West Nile | PCR | Not Detected |
| Virus | | |
| Assay for the Detection of Human | PCR | Not Detected |
| Parvovirus B19 | | |
| Chromosome Analysis | Karyotyping | 46, XX or 46, XY |
| Assay for the Presence of Viral | In Vitro | Not Detected |
| Contaminants | | |
| Test for Presence of Inapparent | In Vivo | Not Detected |
| Viruses | | |
| TEM for Viruses and Retroviruses | TEM | Not Detected |
| Assay for Adeno Associated Virus | PCR | Not Detected |
| Assay for SV40 | PCR | Not Detected |
| Isoenzyme Analysis | Gel | Human |
| | Electrophoresis | |
| Assay for Porcine Adventitious | PCR | Not Detected |
| Agents (9CFR) | | |
| PERT Assay for Retrovirus Detection | PERT | Not Detected |

Cryopreserved MCB units (1-2) are thawed, cultured and expanded in a manner similar to the bone marrow aspirate-MCB cultures. The cells are expanded for two additional rounds at large scale to obtain the final product. By way of non-limiting example, the final harvested product is concentrated using a scalable downstream process called Tangential Flow Filtration (TFF). The concentrated product is washed in the same TFF system using PlasmaLyte A and HSA. The entire culture and downstream processing e.g., concentration and washing is performed in closed system using tube welders and heat sealers. Those skilled in the art will recognize that other downstream processes, including, for example, centrifugation and/or any other suitable process known in the art, may also be utilized.

The MSC population is then packaged into cryogenic vials, frozen to −80° C. in a stepwise manner using a controlled rate freezer, and stored at <−130° C. in vapor phase liquid N2. Moreover, the population is also tested for sterility, mycoplasma, endotoxin, and identity.

Unlike dead end filtration, TFF is an efficient process for retaining and concentrating larger particulates (cells) while removing non-particulates (culture media). In TFF the tangential feed stream efficiently separates cells from culture media without the clogging that occurs in dead end filtration.

A summary of product dose testing is shown below:

| Test and Specifications | Test Procedure | Limits |
|---|---|---|
| Endotoxin | LAL | <5 EU/Kg/hr |
| Sterility | USP <71> | Negative |
| Mycoplasma | FDA PTC | Negative |
| Cell Yield per Vial | Nucleocounter | ≧7 × 10⁶ |
| (Post Cryopreservation), | | |
| Cell Viability | Nucleocounter | ≧70% |
| (Post Cryopreservation), | | |
| Potency | Under development | Under development |
| Extracellular Markers | | |
| CD34 | FACS | ≦10% |
| CD45 | | ≦10% |
| CD90 | | ≧90% |
| CD44 | | ≧90% |
| CD73 | | ≧90% |
| CD105 | | ≧90% |
| HLA-DR | | ≦10% |

Thus, this manufacturing system represents the next generation in cutting edge processes for MSC production. Specifically, it is scalable, performed in a closed culturing system, and free of animal origin products. Moreover, it employs a closed TFF downstream processing system, which preserves cell viability. Likewise, it also uses a closed vialing system

### Methods of Using Mesenchymal Stem Cells

The MSCs can be used to treat or ameliorate conditions including, but not limited to, stroke, multi-organ failure (MOF), AKI of native kidneys, AKI of native kidneys in multi-organ failure, AKI in transplanted kidneys, kidney dysfunction, multi-organ dysfunction and wound repair that refer to conditions known to one of skill in the art. Descriptions of these conditions may be found in medical texts, such as Brenner & Rector's The Kidney, WB Saunders Co., Philadelphia, last edition, 2012, which is incorporated herein in its entirety by reference.

Stroke or cerebral vascular accident (CVA) is a clinical term for a rapidly developing loss of brain function, due to lack of blood supply. The reason for this disturbed perfusion of the brain can be thrombosis, embolism or hemorrhage. Stroke is a medical emergency and the third leading course of death in Western countries. It is predicted that stroke will be the leading cause of death by the middle of this century. These factors for stroke include advanced age, previous stroke or ischemic attack, high blood pressure, atherosclerosis, diabetes mellitus, high cholesterol, cigarette smoking and cardiac arrhythmia with atrial fibrillation. Therefore, a great need exists to provide a treatment for stroke patients.

AKI is defined as an acute deterioration in kidney excretory function within hours or days. In severe AKI, the urine output may be absent or very low. As a consequence of this abrupt loss in function, azotemia develops, defined as a rise of serum creatinine (SCr) and blood urea nitrogen (BUN) levels. SCr and BUN levels are measured repeatedly in patients at risk for or following established AKI. When BUN levels have increased to approximately 10 fold their normal concentration, this corresponds with the development of uremic manifestations due to the parallel accumulation of uremic toxins in the blood. The accumulation of uremic toxins causes bleeding from the intestines, neurological manifestations, most seriously affecting the brain, leading, unless treated, to coma, seizures and death. A normal SCr level is about 1.0 mg/dL, a normal BUN level is about 20 mg/dL. In addition, acid (hydrogen ions) and potassium levels may rise rapidly and dangerously, resulting in cardiac arrhythmias and possible cardiac arrest and death. If fluid intake continues in the absence of urine output, the patient may become fluid overloaded, often resulting in a congested circulation, pulmonary edema and low blood oxygenation, thereby also threatening the patient's survival. One skilled in the art interprets these physical and laboratory abnormalities, and considers the prescription therapy based on the available information.

Clinical evidence of kidney injury may involve an increase in one or more biomarkers selected from serum creatinine (SCr), blood urea nitrogen (BUN), Cystatin C, Beta-trace protein (BTP) (also known as Prostaglandin D Synthase), Podocalyxin, Nephrin, Alpha 1-microglobulin, Beta 2-microglobulin, Glutathione S-transferase, Interleukin-18, Kidney Injury Molecule-1 (KIM-1), Liver-Type Fatty Acid-Binding Protein, Netrin-1, Neutrophil Gelatinase-Associated Lipocalcin (NGAL), and/or N-Acetyl-Beta-D-Glucosaminidase (NAG).

Major causes of intrinsic AKI may include, for example:

tubular injury (e.g., ischemia due to hypoperfusion (i.e., hypovolemia, sepsis, hemorrhage, cirrhosis, congestive heart failure), endogenous toxins (i.e., myoglobin, hemoglobin, paraproteinemia, uric acid), and/or exogenous toxins (i.e., antibiotics, chemotherapy agents, radiocontrast agents, phosphate preparations));

tubulointerstitial injury (e.g., acute allergic interstitial nephritis (i.e., nonsteroidal anti-inflammatory drugs, antibiotics), infections (i.e., viral, bacterial, fungal infections), infiltration (i.e., lymphoma, leukemia, sarcoid), and/or allograft rejection));

glomerular injury (e.g., inflammation (i.e., anti-glomerular basement membrane disease, antineutrophil cytoplasmic autoantibody disease, infection, cryoglobulinemia, membraneoproliferative glomerulonephritis, Immunoglobulin A nephropathy, systemic lupus erythematosus) and/or hematologic disorders (i.e., Henoch-Schönlein purpuria, polyarteritis nodosa Hemolytic uremic syndrome, thrombotic thrombocytopenic purpura, drugs));

renal microvasculature (i.e., malignant hypertension, toxemia of pregnancy, hypercalcemia, radiocontrast agents, scleroderma, drugs); and/or

large vessels (e.g., arteries (i.e., thrombosis, vasculitis, dissection, thromboembolism, atheroembolism, trauma) and veins (i.e., thrombosis, compression, trauma)).

Moreover, causes of prerenal AKI may include, for example:

intravascular volume depletion (e.g., hemorrhage (i.e., trauma, surgery, postpartum, gastrointestinal), gastrointestinal losses (i.e., diarrhea, vomiting, nasogastric tube loss), renal losses (i.e., diuretic use, osmotic dieresis, diabetes insipidus), skin and mucous membrane losses (i.e., burns, hyperthermia), nephrotic syndrome, cirrhosis, or capillary leak); reduced cardiac output (e.g., cardiogenic shock, pericardial diseases (i.e., restrictive, constrictive, tamponade), congestive heart failure, valvular diseases, pulmonary diseases (i.e., pulmonary hypertension, pulmonary embolism), and/or sepsis);

systemic vasodilation (e.g., sepsis, cirrhosis, anaphylaxis, drugs);

renal vasoconstriction (e.g., early sepsis, hepatorenal syndrome, acute hypercalcemia, drugs (i.e., norepinephrine, vasopressin, nonsteroidal anti-inflammatory drugs, angiotension-converting enzyme inhibitors, calcineurin inhibitors), iodinated contrast agents); and/or

increased intraabdominal pressure (e.g., abdominal compartment syndrome).

Post renal causes of AKI may include, for example:

upper urinary tract extrinsic causes (e.g., retroperitoneal space (i.e., lymph nodes, tumors), pelvic or intraabdominal tumors (i.e., cervix, uterus, ovary, prostate), fibrosis (i.e., radiation, drugs, inflammatory conditions), ureteral ligation or surgical trauma, granulomatosis diseases, hematoma);

lower urinary tract causes (e.g., prostate (i.e., benign prostatic hypertrophy, carcinoma, infection), bladder (i.e., neck obstruction, calculi, carcinoma, infection (schistosomiasis)), functional (i.e., neurogenic bladder secondary to spinal cord injury, diabetes, multiple sclerosis, stroke, pharmacologic side effects of drugs (anticholinergics, antidepressants)), urethral (i.e., posterior urethral valves, strictures, trauma, infections, tuberculosis, tumors));

upper urinary tract intrinsic causes (e.g., nephrolithiasis, strictures, edema, debris (i.e., blood clots, sloughed papillae, fungal ball), malignancy).

AKI can occur in clinical settings in a variety of patients, including, for example, AKI in cancer patients, AKI after cardiac surgery, AKI in pregnancy, AKI after solid organ or bone marrow transplantation, AKI and pulmonary disease (pulmonary-renal syndrome), AKI and liver disease, and AKI and nephrotic syndrome. (See Brenner and Rector's, The Kidney, WB Saunders Co., Philadelphia, 9th Edition (2012) (incorporated herein by reference in its entirety).

In addition, those skilled in the art will recognize that endogenous and/or exogenous toxins can cause acute tubular injury.

By way of non-limiting example, endogenous toxins may include, for example, myoglobulinuria; muscle breakdown (e.g., due to trauma, compression, electric shock, hypothermia, hyperthermia, seizures, exercise, burns, etc.); metabolic disorders (e.g., hypokalemia, hypophosphatemia); infections (e.g., tetanus, influenza); toxins (e.g., isopropyl alcohol, ethanol, ethylene glycol, toluene, snake and insect bites, cocaine, heroin); drugs (e.g., hydroxymethylglutaryl-coenzyme A reductase inhibitors, amphetamines, fibrates); inherited diseases (e.g., deficiency of myophosphorylase, phosphofructokinase, carnitine palmityltransferase); autoimmune disorders (e.g., polymyositis, dermatomyositis); hemoglobinuria; mechanical causes (e.g., prosthetic valves, microangiopathic hemolytic anemia, extracorporeal circulation); drugs (e.g., hydralazine, methyldopa); chemicals (e.g., benzene, arsine, fava beans, glycerol, phenol); immunologic disorders (e.g., transfusion reaction); genetic disorders (e.g., glucose-6-phosphate dehydrogenase deficiency, paroxysomal nocturnal hemoglobinuria); hyperuricemia with hyperuricosuria; tumor lysis syndrome; hypoxanthane-guanine phosphoribosyltransferase deficiency; myeloma (e.g., light-chain production); and/or oxalate crystalluria (ethylene glycol).

Likewise, non-limiting examples of exogenous toxins can include, for example, antibiotics; aminoglycosides; amphotericin B; antiviral agents (e.g., acyclovir, cidofovir, indinavir, foscarnet, tenofovir); pentamidine; chemotherapeutic agents; ifosfamide; cisplatin; plicamycin; 5-Fluorouracil; cytarabine; 6-Thioguanine; calcineurin inhibitors; cyclosporin; tacrolimus; organic solvents; toluene; ethylene glycol; poisons; snake venom; paraquat; miscellaneous; radiocontrast agents; intravenous immune globulin; nonsteroidal anti-inflammatory drugs; and/or oral phosphate bowel preparations.

Moreover, as shown below, various common drugs can be classified based in pathophysiologic categories of AKI:

| Pathophysiologic | |
|---|---|
| Category | Drugs |
| Vasoconstriction/ | Nonsteroidal anti-inflammatory drugs |
| Impaired | (NSAIDs), angiotensin converting enzyme |
| Microvasculature | inhibitors, angiotensin receptor blockers, |
| Hemodynamics | norepinephrine, tacrolimus, cyclosporine, |
| (prerenal) | diuretics, cocaine, mitomycin C, estrogen, |
| | quinine, interleukin-2, cyclooxygenase-2 |
| | inhibitors |
| Tubular Cell Toxicity | Antibiotics (e.g., aminoglycosides, |
| | amphotericin B, vancomycin, rifampicin, |
| | foscarnet, pentamidine, cephaloridine, |
| | cephalothin), radiocontrast agents, NSAIDs, |
| | acetaminophen, cyclosporine, cisplatin, |
| | mannitol, heavy metals, intravenous immune |
| | globulin (IVIG), ifosfamide, tenofovir |
| Acute Interstitial | Antibiotics (e.g., ampicillin, penicillin G, |
| Nephritis | methicillin, oxacillin, rifampin in, |
| | ciprofloxacin, cephalothin, sulfonamides), |
| | NSAIDs, aspirin, fenoprofen, naproxen, |
| | piroxicam, phenylbutazone, radiocontrast |
| | agents, thiazide diuretics, phenytoin, |
| | furosemide, allopurinol, cimetidine, |
| | omeprazole |
| Tubular Lumen | Sulfonamides, acyclovir, cidofovir, |
| Obstruction | methotrexate, triamterene, methoxyflurane, |
| | protease inhibitors, ethylene glycol, indinavir, |
| | oral sodium phosphate bowel preparations |
| Thrombotic | Clopidogrel, cocaine, ticlopidine, cyclosporine, |
| Microangiopathy | tacrolimus, mitomycin C, oral contraceptives, |
| | gemcitabine, bevacizumab |
| Osmotic Nephrosis | IVIG, mannitol, dextrans, heat starch |

Multi-organ Failure (MOF) is a condition in which kidneys, lungs, liver and/or heart functions are impaired simultaneously or successively, associated with mortality rates as high as 100% despite the modern medical support. MOF patients frequently require intubation and respirator support because their lungs may develop Adult Respiratory Distress Syndrome (ARDS), resulting in inadequate oxygen uptake and CO₂ elimination. MOF patients may also depend on hemodynamic support, vasopressor drugs, to maintain adequate blood pressures. MOF patients with liver failure may exhibit bleeding along with accumulation of toxins that often impair mental functions. Patients may need blood transfusions and clotting factors to prevent or stop bleeding. It is considered that MOF patients may be given MSC therapy to address AKI and MOF.

Early graft dysfunction (EGD) or transplant associated-acute kidney injury (TA-AKI) is AKI that affects the transplanted kidney in the first few days after implantation. The more severe TA-AKI, the more likely it is that patients will suffer from the same complications as those who have AKI in their native kidneys, as above. The severity of TA-AKI is also a determinant of enhanced graft loss due to rejection(s) in the subsequent years. These are two strong indications for the prompt treatment of TA-AKI with the MSCs of the present invention.

Chronic renal failure (CRF) or Chronic Kidney Disease (CKD) is the progressive loss of nephrons and consequent loss of renal function due to a variety of causes, including diabetic nephropathy and hypertensive nephropathy, resulting in End Stage Renal Disease (ESRD), at which time patient survival depends on dialysis support or kidney transplantation. The need for MSC therapy of the present invention will be determined on the basis of physical and laboratory abnormalities described above.

In some embodiments, the MSCs may be administered to patients in need thereof when one of skill in the art determines that conventional therapy fails. Conventional therapy includes hemodialysis, antimicrobial therapies, blood pressure medication, blood transfusions, intravenous nutrition and in some cases, ventilation on a respirator in the ICU. Hemodialysis is used to remove uremic toxins, improve azotemia, correct high acid and potassium levels, and eliminate excess fluid. In other embodiments of methods of use of MSCs of the invention, the MSCs of the invention are administered as a first line therapy. The methods of use of MSCs of the present invention is not limited to treatment once conventional therapy fails and may also be given immediately upon developing an injury or together with conventional therapy.

In certain embodiments, the MSCs are administered to a subject once. This one dose is sufficient treatment in some embodiments. In other embodiments the MSCs are administered 2, 3, 4, 5, 6, 7, 8, 9 or 10 times in order to attain or sustain a therapeutic effect.

Monitoring patients for a therapeutic effect of the MSCs delivered to a patient in need thereof and assessing further treatment will be accomplished by techniques known to one of skill in the art. For example, renal function will be monitored by determination of SCr and BUN levels, serum electrolytes, measurement of renal blood flow (ultrasonic method), creatinine and insulin clearances, urine output, and other methods. A positive response to therapy for AKI includes return of excretory kidney function, normalization of urine output, blood chemistries and electrolytes, repair of the organ and survival. For MOF, positive responses also include improvement in blood pressure, blood oxygenation, and improvement in functions of one or all organs.

In other embodiments the MSCs are used to effectively repopulate dead or dysfunctional kidney cells in subjects that are suffering from chronic kidney pathology including CKD. The effect may be the results of the paracrine and/or endocrine effects of the MSCs that induce endogenous progenitor cells in the kidney. Additionally (or alternatively), this effect may be because of the “plasticity” of the MSC populations. The term “plasticity” refers to the phenotypically broad differentiation potential of cells that originate from a defined stem cell population. MSC plasticity can include differentiation of stem cells derived from one organ into cell types of another organ. “Transdifferentiation” refers to the ability of a fully differentiated cell, derived from one germinal cell layer, to differentiate into a cell type that is derived from another germinal cell layer.

It was previously assumed that stem cells gradually lose their pluripotency and thus their differentiation potential during organogenesis. It was thought that the differentiation potential of somatic cells was restricted to cell types of the organ from which respective stem cells originate. This differentiation process was thought to be unidirectional and irreversible. However, recent studies have shown that somatic stem cells maintain some of their differentiation potential. (See Homback-Klonich et al., J Mol Med (Berl) 86(12):1301-1314 (2008)). For example, stem cells may be able to transdifferentiate into muscle, neurons, liver, myocardial cells, and kidney. It is possible that as yet undefined signals that originate from injured and not from intact tissue act as transdifferentiation signals.

In certain embodiments, a therapeutically effective dose of MSCs is delivered to the patient. An effective dose for treatment will be determined by the body weight of the patient receiving treatment, and may be further modified, for example, based on the severity or phase of the stroke, kidney or other organ dysfunction, for example the severity of AKI, the phase of AKI in which therapy is initiated, and the simultaneous presence or absence of MOF. In some embodiments of the methods of use of the MSCs of the invention, from about 1×10⁵ to about 1×10¹° MSCs per kilogram of recipient body weight are administered in a therapeutic dose. Preferably from about 1×10⁵ to about 1×10⁸ MSCs per kilogram of recipient body weight is administered in a therapeutic dose. More preferably from about 7×10⁵ to about 5×10⁸ MSCs per kilogram of recipient body weight is administered in a therapeutic dose. More preferably from about 1×10⁶ to about 1×10⁸ MSCs per kilogram of recipient body weight is administered in a therapeutic dose. More preferably from about 7×10⁵ to about 7×10⁶ MSCs per kilogram of recipient body weight is administered in a therapeutic dose. More preferably about 2×10⁶−5×10⁶ MSCs per kilogram of recipient body weight is administered in a therapeutic dose. The number of MSCs used will depend on the weight and condition of the recipient, the number of or frequency of administrations, the route of administration, and other variables known to those of skill in the art. For example, a therapeutic dose may be one or more administrations of the therapy.

The therapeutic dose of MSCs is administered in a suitable solution for injection (i.e., infusion or bolus). Solutions are those that are biologically and physiologically compatible with the cells and with the recipient, such as buffered saline solution, PlasmaLyte or other suitable excipients, known to one of skill in the art.

In certain embodiments of the MSCs of the invention are administered to a subject at a rate between approximately 0.5 and 1.5 mL of MSCs in physiologically compatible solution per second. Preferably, the MSCs of the invention are administered to a subject at a rate between approximately 0.83 and 1.0 mL per second. More preferably, the MSCs are suspended in approximately 100 mL of physiologically compatible solution and are completely injected into a subject between approximately one and three minutes. More preferably the 100 mL of MSCs in physiologically compatible solution is completely injected in approximately one minute.

In other embodiments, the MSCs are used in trauma or surgical patients scheduled to undergo high-risk surgery such as the repair of an aortic aneurysm. In the case of poor outcome, including infected and non-healing wounds, development of MOF post-surgery, the patient's own MSCs, prepared according to the methods of the invention, that are cryopreserved may be thawed out and administered as detailed above. Patients with severe AKI affecting a transplanted kidney may either be treated with MSCs, prepared according to the methods of the invention, from an unrelated donor or the donor of the transplanted kidney (allogeneic) or with cells from the recipient (autologous). Allogeneic or autologous MSCs, prepared according to the methods of the invention, are an immediate treatment option in patients with TA-AKI and for the same reasons as described in patients with AKI of their native kidneys.

In certain embodiments, the MSCs of the invention are administered to the patient by infusion intravenously or intra-arterially (for example, for renal indications, via femoral artery into the supra-renal aorta). Preferably, the MSCs of the invention are administered via the supra-renal aorta. In certain embodiments, the MSCs of the invention are administered through a catheter that is inserted into the femoral artery at the groin. Preferably, the catheter has the same diameter as a 12-18 gauge needle. More preferably, the catheter has the same diameter as a 15 gauge needle. The diameter is relatively small to minimize damage to the skin and blood vessels of the subject during MSC administration. Preferably, the MSCs of the invention are administered at a pressure that is approximately 50% greater than the pressure in the subject's aorta. More preferably, the MSCs of the invention are administered at a pressure of between about 120 and 160 psi. Generally, at least 95% of the MSCs of the invention survive injection into the subject. Moreover, the MSCs are generally suspended in a physiologically acceptable carrier containing about 5% HSA. The HSA, along with the concentration of the cells prevents the MSCs from sticking to the catheter or the syringe, which also insures a high (i.e. greater than 95%) rate of survival of the MSCs when they are administered to a subject. The catheter is advanced into the supra-renal aorta to a point approximately 20 cm above the renal arteries. Preferably, blood is aspirated to verify the intravascular placement and to flush the catheter. More preferably, the position of the catheter is confirmed through a radiographic or ultrasound based method. Preferably the methods are transesophageal echocardiography (TEE) or an X-ray. The MSCs of the invention are then transferred to a syringe that is connected to the femoral catheter. The MSCs, suspended in the physiologically compatible solution are then injected over approximately one to three minutes into the patient. Preferably, after injection of the MSCs of the invention, the femoral catheter is flushed with normal saline. Optionally, the pulse of the subject found in the feet is monitored, before, during and after administration of the MSCs of the invention. The pulse is monitored to ensure that the MSCs do not clump during administration. Clumping of the MSCs can lead to a decrease or loss of small pulses in the feet of the subject being administered MSCs.

In certain embodiments, a therapeutically effective dose of MSCs is delivered intravenously (IV) to the patient. The therapeutic dose of MSCs in a suitable solution for injection is administered via IV injection, infusion, or bolus or other suitable methods into a peripheral, femoral, jugular, or other vein known to one of ordinary skill in the art.

### Dose Rationale

A dose of 2×10⁶ human MSCs (hMSC)/kg of a preparation of human MSC designed for clinical use has been selected for further investigation of the preparation in clinical studies of AKI. Data from a Phase 1 study, other clinical investigations of hMSC, as well as nonclinical investigations support selection of this dose.

The Phase 1 study evaluated three doses levels of PL-produced hMSC, designated AC607, including 7×10⁵, 2×10⁶ and 7×10⁶ hMSC/kg. All doses of AC607 were safe and well tolerated in this study, with no treatment related adverse events or serious adverse events observed in any dose cohort. In other clinical studies, hMSC have been administered to subjects across a range of doses with no reported safety issues. Doses of hMSC in these other studies have typically ranged from 150 to 300 million MSC per subject (approximately 2 to 4×10⁶ MSC/kg for a 70-kg subject), consistent with the selected dose. (See Ankrum et al., Trends Mol. Med. 16(5):203-09 (2010)). Moreover, published data suggest that hMSC doses of at least 1×10⁶ MSC/kg are pharmacologically active in non-AKI clinical indications. (See Hare et al., J. Am. Coll Cardiol 2227-86 (2009)).

In a rat I/R model of AKI, hMSC at an intra-arterial dose of 1×10⁶ hMSC/kg significantly reduced serum creatinine (SCr) when administered after a 7-fold increase in SCr. (See Cao et al., Biotechnol Lett 32:725-32 (2010)). Consistent with data for hMSC, a nonclinical study demonstrated that intra-arterial administration of rat MSC (rMSC) significantly lowered SCr in the rat AKI model at doses of 2×10⁶ rMSC/kg or 5×10⁶ rMSC/kg, but not at 0.5×10⁶ rMSC/kg. (See Tögel et al., Stem Cells Dev 18:475-85 (2009)). Further, another nonclinical investigation demonstrated that a single intra-arterial injection of rMSC at doses up to 15×10⁶ rMSC/kg was well tolerated in rats with AKI.

Collectively, these clinical and nonclinical data support selection of 2×10⁶ MSC/kg of AC607 as a safe and pharmacologically active dose for future clinical studies of AKI.

### Clinical Data

In the Phase 1 study, a single intra-arterial injection of AC607 at 7×10⁵ hMSC/kg, 2×10⁶ hMSC/kg, or 7×10⁶ hMSC/kg was safe and well tolerated in 16 subjects undergoing elective cardiac surgery who were at risk for developing postoperative AKI.

In summary, a single, intra-arterial dose of up to 7×10⁶ hMSC/kg of AC607 was safe and well tolerated when administered to subjects after cardiac surgery.

Currently, there are 158 clinical studies of hMSC (not limited to AKI trials) currently listed on ClinicalTrials.gov. In these clinical investigations, hMSC doses most commonly range from 2×10⁶ MSC/kg to 4×10⁶ MSC/kg. (See Ankrum et al., Trends Mol Med 16(5):203-209 (2010)). Moreover, hMSC have been safely administered to subjects at doses of up to 8×10⁶ MSC/kg with no reported treatment related adverse events. (See Kebriaei et al., Biol Blood Marrow Transplant. 15:804-11 (2009)).

In a double-blind, placebo-controlled study of 60 patients with acute myocardial infarction, subjects were randomized 2:1 to receive either hMSC or placebo. (See Hare et al., J Am Coll Cardiol 54:2227-86 (2009)). hMSC were administered at doses of 0.5×10⁶ MSC/kg, 1.6×10⁶ MSC/kg, or 5×10⁶ MSC/kg. The rate of arrhythmias was 4-fold less subjects that received hMSC compared to the placebo group (8.8% versus 36.8%, P=0.025). hMSC-treated subjects experienced fewer premature ventricular contractions (PVC) compared to those treated with placebo (P=0.017), and the percentage of patients that experienced more than 10 PVC per hour was significantly reduced in hMSC-treated compared to placebo-treated subjects (10.0% versus 24%, P=0.001). Interestingly, the rate of PVC exhibited a dose-response effect with reductions in PVC detected in the 1.6×10⁶ MSC/kg and 5×10⁶ MSC/kg groups but not in the 0.5×10⁶ MSC/kg group, compared to the placebo group.

The invention will be further illustrated in the following non-limiting examples.

### EXAMPLES

### Example 1

### DNA Isolation from Human Blood Samples

The objective of this Example is to ensure that a sufficient quantity of DNA is isolated from human blood samples using the Qiagen DNeasy Blood and Tissue Kit for subsequent determination of the GT repeat lengths in both HO-1 promoter alleles. This protocol is designed for use in the isolation of total DNA from human blood samples. DNA samples are sent to an outside vendor for fragment length analysis to determine the GT repeat lengths in the HO-1 promoter region.

Required Materials
1. Anti-coagulated human blood in and EDTA-vacutainer (from a refrigerated or a thawed, frozen sample)
2. Qiagen DNeasy Blood & Tissue Kit (Cat. #69504)
   Proteinase K
   Buffer AL
   Buffer AW 1
   Buffer AW2
   Buffer AE
   Spin Columns
   Collection Tubes
3. Ethanol (96-100%)
4. Water bath set to 56° C.
5. 1.5 mL microcentrifuge tubes
6. Phosphate-buffered saline (PS), Lonza catalog #17-513F (or equivalent)
7. Assorted serological pipettes

25 mL ethanol was added to Buffer AW and 30 mL ethanol was added to Buffer AW2 prior to procedure. All centrifugations were performed at room temperature. Four separate DNeasy columns were used for each donor's blood sample, and the 4 DNA samples purified from the same donor were combined at the end of the purification procedure.

Procedure
1. For each blood sample, 4 microcentrifuge tubes were with the blood sample identification.
2. 20 μL proteinase K were added to each of the 4 microcentrifuge tubes. The blood sample vacutainer tube was thoroughly mixed by vortexing and 100 μL anti-coagulated blood was transferred to each microcentrifuge tube, then 100 μL PBS was added to each microcentrifuge tube.
3. Vacutainer tube was capped and wrapped with parafilm. The remaining blood was stored in the freezer.
4. 200 μL, Buffer AL was added to each microcentrifuge tube and mixed thoroughly by vortexing. Tubes were incubated at 56° C. for 10 minutes.
5. 200 μL ethanol (96-100%) were added to each tube and mixed thoroughly by vortexing.
6. The mixture was pipette from each tube into a separate DNeasy Mini spin column placed in a 2 mL collection tube. Tubes were centrifuged for 1 min at ≧6000×g. Flow-through and collection tube were discarded.
7. Each spin column was placed in a fresh 2 mL collection tube. 500 μL Buffer AW1 was added to each spin column. Tubes were centrifuged for 1 min at ≧6000×g. Flow-through and collection tube were discarded.
8. Each spin column was placed in a fresh 2 mL collection tube. 500 μL Buffer AW2 was added to each spin column. Tubes were centrifuged for 3 min at ≧20,000×g (14,000 rpm). Flow-through and collection tube were discarded.
9. Each spin column was transferred to a fresh 1.5 mL micro-centrifuge tube. DNA was eluted by adding 200 μL Buffer AE to the center of each spin column membrane. Tubes were incubated for 1 minute at room temperature (15-25° C.) and were centrifuged for 1 minute at ≧6000×g.
10. The 4 DNA samples purified from the same donor were combined into a single 1.5 L microcentrifuge tube.
11. The purified DNA was quantitated by measuring the optical density (OD) 260.
   a. 20 μL of the combined DNA sample was diluted with 80 μL of water in a fresh 1.5 mL tube.
   b. the diluted DNA was pipette into a well of a 96-well UV compatible plate.
   c. the OD at 260 and 280 nanometers was measured.
   d. the formula of OD_{260/280} of 1=50 μg/mL DNA was used
      i. For example, an OD_{260/280} of 0.015=0.75 μg/mL DNA
   e. the DNA concentration was confirmed using the nanodrop method, if available.
12. DNA sample tube was stored at −20° C.
13. Date of DNA isolation was recorded.
14. A sufficient quantity of DNA was submitted for fragment analysis. The GT repeat length was determined by comparing the resulting fragment size to the published HO-1 promoter sequence and fragment sizes of synthetic DNA fragments with known GT repeat lengths.

### Example 2

### DNA Isolation from Cryopreserved MSC

The objective of this Example is to ensure that a sufficient quantity of DNA is isolated from cryopreserved MSC samples using the Qiagen DNeasy Blood and Tissue Kit for subsequent determination of the GT repeat lengths in both alleles of the HO-1 promoter. This protocol is designed for use in the isolation of total DNA from frozen MSC samples. DNA samples are sent to an outside vendor for DNA fragment length analysis to determine the GT repeat lengths in the HO-1 promoter region. For example, MSCs may come from an MCB.

Required Materials

### 1. Cryopreserved MSC

### 2. Qiagen DNeasy Blood & Tissue Kit (Cat. #69504)

Proteinase K
Buffer AL
Buffer AW 1
Buffer AW2
Buffer AE
Spin Columns
Collection Tubes

### 3. Ethanol (96-100%)

4. Water bath set to 56° C.
5. 1.5 mL microcentrifuge tubes
6. Phosphate-buffered saline (PBS), Lonza catalog #17-513F (or equivalent)
7. Assorted serological pipettes

25 mL ethanol was added to Buffer AW and 30 mL ethanol was added to Buffer AW2 prior to procedure. All centrifugations were performed at room temperature.

Procedure
1. A frozen MSC sample (approximately 1×10⁵ to 5×10⁶ MSC) was thawed in a 37° C. water bath and the cells were transferred to a 1.5 mL microcentrifuge tube. Cells were spun for 1 minute at 6000×g (8000 rpm). Supernatant was aspirated and 200 μl PBS was added, mixed, and then 20 μL Proteinase K was added.
2. 200 μL Buffer AL was added and mixed thoroughly by vortexing. Tubes were incubated at 56° C. for 10 minutes.
3. 200 μL ethanol (96-100%) was and mixed thoroughly by vortexing.
4. The mixture was pipetted into a DNeasy Mini spin column placed in a 2 mL collection tube and centrifuged for 1 min at ≧6000×g. Flow-through and collection tube were discarded.
5. The spin column was placed in a fresh 2 mL collection tube. 500 μL Buffer AW1 was added and tube was centrifuged for 1 min at ≧6000×g. Flow-through and collection tube were discarded.
6. Spin column was placed in a fresh 2 mL collection tube. 500 μL Buffer AW2 was added and tube was centrifuged for 3 min at ≧20,000×g (14,000 rpm). Flow-through and collection tube were discarded.
7. Spin column was transferred to a fresh 1.5 mL micro-centrifuge tube. DNA was eluted by adding 200 μL Buffer AE to the center of the spin column membrane and tube was incubated for 1 minute at room temperature (15-25° C.) and centrifuged for 1 minute at ≧6000×g.
8. DNA was quantitated by measuring the optical density (OD) 260.
   a. 20 μL of the DNA sample was diluted with 80 μL of water in a fresh 1.5 mL tube.
   b. the diluted DNA was pipette into a well of a 96-well UV compatible plate.
   c. the OD at 260 and 280 nanometers was measured.
   d. the formula of OD_{260/280} of 1=50 μg/mL DNA was used
      i. For example, an OD_{260/280} of 0.015=0.75 μg/mL DNA
   e. the DNA concentration was confirmed using the nanodrop method, if available.
9. DNA was stored at −20° C.
10. A sufficient quantity of DNA was submitted for fragment analysis. The GT repeat length was determined by comparing the resulting fragment size to the published HO-1 promoter sequence and fragment sizes synthetic DNA fragments with known GT repeat lengths.

### Example 3

### Human HO-1 Gene Promoter GT Repeat Analysis

The objective of this example is to determine the number of GT repeats in the human HO-1 gene promoter using DNA fragment length analysis. Total DNA purified from human blood (see Example 1, supra) or MSC samples (see Example 2, supra) were submitted to an outside vendor (University of Utah Genetics Core Facility) for fragment length analysis. Polymerase chain reaction (PCR) using a specific, forward oligonucleotides primer labeled with 6-fluorescein amidite (6-FMA) and a specific, unlabeled reverse primer flanking the GT-repeats within the HO-1 promoter were used to synthesize 6-FAM labeled DNA fragments. Fragment length analysis of the 6-FAM labeled PCR products were conducted by the outside vendor to determine the number of GT repeats in the HO-1 promoter region.

Required Materials
1. Total DNA purified from blood or cells using DNeasy kit
   50-100 ng per sample is needed.
2. Control DNA from Master Cell Bank (MCB) 808 or MCB 810 (50-100 ng per sample).
3. Reverse-phase HPLC purified 6-FAM labeled forward primer, synthesized and labeled by integrated technologies (IDT)
   forward primer sequence 5′-6-FAM-TGACATTTTAGGGAGCTGGAGACA (SEQ ID NO:1)
   the forward primer will be diluted to a 10 μM solution and used as 1 μL per 20 μL PCR reaction.
4. Reversed-phase HPLC purified unlabeled reverse primer
   reverse primer sequence 5-′ACAAAGTCTGGCCATAGGAC (SEQ ID NO:2)
   the reverse primer will be diluted to a 10 μM solution and used as 1 μL per 20 μL PCR reaction.
5. Microcentrifuge tubes (1.5 mL)

DNA purified from human blood or MSC samples using Qiagen's DNeasy blood and tissue kit #69504 were used. For positive controls, DNA from MCB 808 or other samples, such as synthetic DNA with known fragment lengths using the same PCR primers were submitted.

Procedure
1. 50-100 ng of total DNA from each sample to be genotyped (or positive control DNA) were aliquoted into separate 1.5 mL microcentrifuge tubes.
2. 50 μL of the 50 μM forward and reverse primer stock solutions were aliquoted into separate 1.5 mL microcentrifuge tubes. The primers were diluted to a 10 μM working solution and were used at 1 μL PCR reactions at the external vendor.
3. The DNA samples and primer stock solutions were submitted to the external vendor.
4. Any remaining volume of the primers remained at the vendor for future PCR and fragment length analysis.

Data Analysis
1. Fragment length data received from external vendor.
2. Confirmed that the positive control (e.g., MCB 808 and 810) fragments were the expected length (in base pairs), as predicted from the published HO-1 promoter sequence.
3. Fragment sizes (in base pairs) were determined for submitted DNA samples from the plots received from the vendor.
4. Sizes of fragments and numbers of GT repeats for each sample were recorded.

A fragment Conversion Table is shown in FIG. 1B.

### Example 4

### Preparation of PL

A MSC expansion medium containing PL was developed as an alternative to FBS. PL isolated from platelet rich plasma (PRP) were analyzed with either Human 27-plex (from BIO-RAD) or ELISA to show that inflammatory and anti-inflammatory cytokines as well as a variety of mitogenic factors are contained in PL, as shown below in Table 1. The human-plex method presented the concentration in [pg/mL] from undiluted PL while in the ELISA the PL was diluted to a thrombocyte concentration of 1×10⁹/mL and used as 5% in medium (the values therefore have to be multiplied by at least 20). <: below the detection limit. Values with a black background are anti-inflammatory cytokines and cells with a gray background are inflammatory cytokines.

TABLE 1
Determination of factor-concentrations in PL.

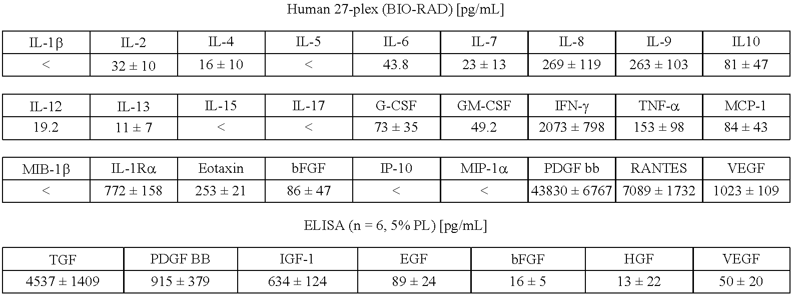

For effective expansion of MSC, an optimized preparation of PL is needed. The protocol includes pooling PRPs from at least 10 donors (to equalize for differences in cytokine concentrations) with a minimal concentration of 3×10⁹ thrombocytes/mL.

PL was prepared either from pooled platelet concentrates designed for human use (produced as TK5F from the blood bank at the University Clinic UKE Hamburg-Eppendorf, pooled from 5 donors) or from 7-13 pooled buffy coats after centrifugation at 200×g for 20 min. PRP was aliquoted into small portions, frozen at −80° C., thus producing PL which is thawed immediately before use. PL-containing medium was prepared fresh for each cell feeding. Medium contained αMEM as basic medium supplemented with 5 IU Heparin/mL medium (source: Ratiopharm) and 5% of freshly thawed PL.

### Example 5

### Production of MSCs in PL-Supplemented Media

Bone marrow was collected from non-mobilized healthy donors. White blood cells (WBC) concentrations and CFU-F (colony forming units—fibroblasts) from bone marrow isolated from different donors varied. Data are summarized in Table 2, below.

TABLE 2
Comparison of Different Bone Marrow Donors

| | | | WBC per 50 ml | | |
|---|---|---|---|---|---|
| Donor | Sex | Age | [×10⁸] | Physician | CFU-F/10⁶ cells |
| 1 | M | 60+ | 19.1 | FA | 16 |
| 2 | M | 50+ | 10.1 | AZ | >250 |
| 3 | M | 50+ | 3.1 | AZ | 0.2 |
| 4 | F | | 6.6 | AZ | 50 |
| 5 | M | 37 | 6.4 | Clinical | 60 |
| 6 | M | 29 | 12.1 | NK | 250 |
| 7 | M | | 6.9 | AZ | 62 |
| 8 | F | 40 | 16.8 | FA | 230 |
| 9 | F | 24 | 12.7 | FA | 43 |
| 10 | F | 37 | 11.6 | FA | 225 |
| 11 | M | 24 | 21.1 | FA | 260 |
| 12 | F | 26 | 4.6 | AZ | 47 |
| 13 | F | 25 | 10.1 | FA | 23 |
| 14 | M | | 17.4 | FA | 12 |
| 15 | W | 28 | 11.1 | FA | 130 |

Once the bone marrow was received, a sample was removed and sent for infectious agent testing. Testing includes human immunodeficiency virus, type 1 and 2 (HIV I/II), human T cell lymphotrophic virus, type I and II (HTLV I/II), hepatitis B virus (HBV), hepatitis C virus (HCV), Treponema pallidum (syphilis) and cytomegalovirus (CMV).

Reagents used are shown in Table 3, below.

TABLE 3
Reagents.

| | Final | | FDA- | | | |
|---|---|---|---|---|---|---|
| Reagent | Concentration | Source | Approved | Vendor | Cat # | COA |
| AlphaMEM | Trace amounts | Non- | Yes | Lonza | 12-169F | Yes |
| | | mammalian | | | | |
| Platelet Rich | Trace amounts | Human | No | American Red | NA | No |
| Plasma | | | | Cross | | |
| 25% Human | 5% | Human | Yes | NDC 0053- | NA | Yes |
| Serum | | | | 7680-32 | | |
| Albumin | | | | | | |
| PlasmaLyte A | 40 mL | Non- | Yes | Baxter | 2B2543Q | Yes |
| | | mammalian | | | | |
| Phosphate | Trace amounts | Non- | Yes | Lonza | | Yes |
| Buffered | | mammalian | | | | |
| Saline | | | | | | |
| Trypsin/EDTA | Trace amounts | Recombinant | Yes | Roche/Lonza | | Yes |
| L-Glutamine | Trace amounts | Non- | No | Lonza | | Yes |
| | | mammalian | | | | |
| DMSO | More than | Non- | No | Protide | PP1300 | Yes |
| | Trace amounts | mammalian | | Pharmaceutical | | |

300 μL of whole bone marrow was plated in 15 mL of αMEM media containing 5% PL in tissue culture flask with 75 cm² of growth area or in larger vessels for 2-10 days to allow the MSCs to adhere. Residual non-adherent cells were washed from the flask. αMEM media containing 5% platelet-rich plasma was added to the flask. Cells were allowed to grow until 70% confluency (approximately 3-4 days). Cells were then trypsinized and re-plated into a Nunc Cell Factory™. Cells remained in the Cell Factory™ for approximately 6-8 days for expansion with media exchanges every 4 days.

Cells were harvested by first washing in phosphate buffered saline (PBS), treating with trypsin and washing with αMEM and then cryopreserved in 10% DMSO, 5% HSA in PlasmaLyte A using controlled-rate freezing. When the cells were required for infusion, they were thawed, washed free of DMSO and resuspended to the desired concentration in PlasmaLyte A containing 5% HSA.

The final cell product consisted of approximately 10⁶-10⁸ cells per kg of weight of the subject (depending on the dose schedule) suspended in 50 mL PlasmaLyte A with 5% HSA. No growth factors, antibodies, stimulants, or any other substances were added to the product at any time during manufacturing. The final concentration was adjusted to provide the required dose such that the volume of product that is returned to the patient remained constant.

### Example 6

### Comparison of MSCs Grown in PL- and FBS-Supplemented Media

The expansion of MSCs from bone marrow (BM) has been shown to be more effective with PL- compared to FBS-supplemented media. The size, as well as the number, (Table 4), of CFU-F were considerably higher using PL as supplement in the medium (see WO2010/017216, incorporated herein by reference).

TABLE 4
CFU-F from MSCs with FBS- or PL-supplemented media.
Values are shown for 10⁷ plated cells.

| | αMEM + FBS | αMEM + PL |
|---|---|---|
| mean ± SE | 415 ± 97 | 1181 ± 244 |

MSCs were isolated by plating 5×10⁵ mononuclear cells/well in 3 mL. The more effective isolation of MSCs with PL-supplemented media is followed by a more rapid expansion of these cells over the whole cultivation period until senescence.

Also, MSCs cultured in PL-supplemented media are less adipogenic in character when compared to MSCs cultured in FBS-supplemented media.

MSC have been described to act in an immunomodulatory fashion by impairing T-cell activation without inducing anergy. A dilution of this effect has been shown in vitro in mixed lymphocyte cultures (MLC) leading eventually to an activation of T-cells if decreasing amounts of MSC are added to the MLC reaction. This activation process is not observed when PL-generated MSC are used in the MLC as the third party. MSCs are less immunogenic after PL-expansion whereas FBS seems to act as a strong antigen or at least has adjuvant function in T-cell stimulation. This result is also reflected in differential gene expression showing a down-regulation of MHC II compounds.

Additional data from differential gene expression analysis of PL-generated compared to FBS-generated MSC showed an up-regulation of genes involved in the cell cycle (e.g. cyclins and cyclin dependent kinases) and the DNA replication and purine metabolism. On the other hand, genes functionally active in cell adhesion/extracellular matrix (ECM)-receptor interaction, differentiation/development, TGF-β signaling and thrombospondin induced apoptosis could be shown to be down-regulated in PL-generated MSC, further supporting the results of faster growth and accelerated expansion.

Furthermore, evidence demonstrates that MSCs grown in PL-supplemented medium are more protective against ischemia-reperfusion damage than MSCs grown in FBS-supplemented medium. Human kidney proximal tubular cells (HK-2) were forced to start apoptotic events by incubation with antimycin A, 2-deoxyclucose and calcium ionophore A23187 (Lee et al., J Am Soc Nephrol 13, 2753-2761 (2002); Xie et al., J Am Soc Nephrol 17, 3336-3346 (2006)). This treatment chemically mimics an ischemic event. Reperfusion was simulated by refeeding the HK-2 cells with rescue media consisting of conditioned medium incubated for 24 h on confluent layers of MSCs grown with either αMEM+10% FBS or αMEM+5% PL.

Supernatants from MSCs grown in PL-containing medium are more effective in reducing HK-2 cell death after chemically simulated ischemia/reperfusion than supernatants from MSCs grown in FBS-supplemented medium.

A parallel FACS assay detecting annexin V that binds to apoptotic cells showed similar results. The proportion of viable cells (=annexin V negative) was higher in the HK-2 cells rescued with MSC-conditioned PL medium (85.7%, as compared to 78.0% in MSC-conditioned FBS medium. Thus, it appears that PL-MSCs contain a higher rate of factors that prevent kidney tubular cells from dying after ischemic events and/or less factors that promote cell death compared to FBS-MSC conditioned medium. Thus, PL appears to be the supplement of choice to expand MSCs for the clinical treatment of ischemic injury.

### Example 7

### Cryopreservation Protocol for hMSCs

Mesenchymal stem cells were cryopreserved in a DMSO solution, at a final concentration of 10%, for long-term storage in vapor phase liquid nitrogen (LN2, <−150° C.). The viability and functionality of hMSCs in prolonged storage has been demonstrated and there is currently no recognized expiration of products that remain in continuous LN2 storage.

hMSCs were derived from human bone marrow.

Reagents, Standards, Media, and Special Supplies Required:

DMSO (Protide Pharmaceuticals)

HSA (NDC 0053-7680-32)

PlasmaLyte A

Cryovials

Dispensing Pin

20 cc Syringe without Needle

30 cc Syringe without Needle

18 gauge Blunt Fill Needle

Alcohol Preps

Betadine Preps

Ice Bucket

10 mL serological pipette

25 mL serological pipette

250 mL Conical Tube

Cryogloves

Instrumentation:

Pipettes

Biological Safety Cabinet (BSC)

Controlled Rate Freezer (CRF)

LN2 Storage Freezer with Inventory System

Centrifuge

A. Calculate the Number of Cyrovials Needed to Freeze the hMSC Product
1. Calculating Freeze Mix: The number of cryovials necessary to freeze a give quantity of cells was calculated. The MSCs are stored at 15×10⁶/mL. Thus, the number of cells present was divided by this number to ascertain the volume of cells and medium to be frozen.

For example, 3.71×10⁸=24.7 mL.
2. Calculating number of cryovials: The number of vials needed for a given volume of cells plus medium was calculated. The volume of the cryovials was 1 mL or 4 mL. Thus, the volume calculated above was divided into the number of cryovials needed.

For example: 24 mL=6, 4 mL cyrovials

B. Calculate the Total Freeze Volume

Total freeze volume consisted of 10% DMSO by volume, 20% albumin by volume, and the remaining volume PlasmaLyte (70%).

For example: Total Freeze Volume=24 mL
DMSO=2.4 mL
Albumin=4.8 mL
PlasmaLyte=16.8 mL

C. Prepare Freeze Mix

1. Ice bucket prepared.
2. The desired volume of DMSO was obtained with an appropriate sized syringe.
3. The same volume of PlasmaLyte that was obtained.

a. e.g. 6 mL of DMSO, 6 mL of PlasmaLyte

4. The DMSO and PlasmaLyte were added to the “Freeze Mix” tube.
5. The solution was mixed and placed on ice to chill for at least 10 minutes.
6. The albumin was placed on ice

D. Prepare sample for freezing
1. The final product was centrifuged in a 250 mL conical tube at 600×g (−1600 rpm) for 5 minutes, no brake.
2. The supernatant was removed to one inch above the cell pellet using a 25 mL serological pipette. The cell pellet was not disturbed.
3. The supernatant was removed and placed in a sterile 250 mL conical tube labeled “Sup”.
4. Both the cells and supernatant were placed on ice

E. Freezing
1. The amount of PlasmaLyte still needed for the freeze mix was calculated and the desired volume was obtained.
   a. For example, the volume of DMSO+the volume of already added PlasmaLyte+the volume of albumin+ell pellet volume minus the total freeze volume equals amount of PlasmaLyte needed.
2. The albumin bag was aseptically spiked with a dispensing pin and the desired volume of albumin was removed.
3. The albumin and PlasmaLyte were added to the “Freeze Mix” tube and mixed.
4. Using a 10 mL serological pipette the chilled freeze mix aseptically removed and added slowly to the resuspended cells. While adding the freeze mix cells were gently mixed by swirling. Once the Freeze Mix was added to the product, the freeze was initiated within 15 minutes. If a delay was expected, the product mixture was placed back on ice. Under no circumstances was the mix allowed to be unfrozen for more than 30 minutes.
5. The lid was placed on the tube containing cell mix and the tube was inverted several times to mix the contents.
6. Using a 10 mL serological pipette the freeze volume was aseptically removed and the appropriate volume was dispensed into each labeled cryovial. In 1.8 mL vials 1 mL of cell mix was placed. In 4.5 mL vials 4 mL of cell mix was placed.
7. The cryovials were then immediately placed on ice and then frozen using the controlled rate freezer to −80° C.

F. Expected Ranges for MSCs Thawed after being Frozen According to Protocol:

### 1. Thawed Product Viability≧70%

### 2. Sterility Testing=Negative

3. Differentiation=growth for adipogenic, osteogenic, and chondrogenic
4. Flow cytometry

a. CD105 (≧90%)

b. CD 73 (≧90%)

c. CD 90 (≧90%)

d. CD44 (≧90%)

e. CD 34 (<10%)

f. CD 45 (<10%)

g. HLA-DR (<10%)

5. Endotoxin<5.0EU/kg body weight
6. Mycoplasma=negative

### Example 8

### Thawing Protocol for hMSCs

Stored hMSC are cryopreserved using DMSO as a cell cryoprotectant. When thawed, DMSO creates a hypertonic environment that leads to sudden fluid shifts and cell death. To limit this effect, the cryopreserved hMSC were washed with a hypertonic solution ameliorating DMSO's unfavorable effects. Post-thaw product release testing was done to ensure processing was performed so as to prevent contamination or cross-contamination.

Reagents, Standards, Media, and Special Supplies Required:

HSA 25% (NDC 52769-451-05)

PlasmaLyte A

Trypan Blue

300 mL Transfer Pack

15 mL conical tube

50 mL conical tube

250 mL Conical Tube

150 mL Transfer Pack

Sterile Transfer Pipette

1.5 mL Eppendorf tube

Red Top Vacutainer Tubes or equivalent

10 cc syringe

20 cc syringe

30 cc syringe

60 cc syringe

5 mL serological pipette

10 mL serological pipette

Ice Bucket

Blunt End Needle

200-1000 μL sterile tips

Cryogloves

Biohazard Bag

Iodine

Alcohol wipes

Instrumentation:

Biological Safety Cabinet (BSC)

Centrifuge

Sterile Connecting Device

Microscope, Light

Thermometer

Water Bath

Hemacytometer

Pipettes

Computer with Freezerworks

Ambient Shipper

A. Wash Solution Preparation
1. The cell dose required for infusion was calculated based on the recipient's weight. The required number of cells for infusion based on recipient weight was calculated by multiplying the cell dosage per kg times the recipient weight in kg to arrive at the number of cells necessary.
2. The number of cryovials needed to achieve the calculated cell dose was then determined.
   a. 1 mL of cell mix contains 15×10⁶ cells.

3. The wash solution volume needed to thaw all required cryovials was then calculated:
For the example below, all numbers listed below are for a 100 kg patient.
a. Volume of product, multiplied times 4 in addition to 80 mL for cell resuspension and testing
   1) for a dose of 7×10⁵ cells=˜7 mL of product thawed and a wash solution volume of 108 mL was used;
   2) for a dose of 2×10⁶ cells=˜19 mL of product thawed and a wash solution volume of 156 mL was used;
   3) for a dose of 5×10⁶ cells=˜46 mL of product thawed and a wash solution volume of 264 mL was used.
b. Wash Solution=20% by volume stock albumin (25% Human, USP, 12.5 g/50 mL), 80% PlasmaLyte
4. A female end was sterile connected to a 300 mL transfer pack.
5. Using sterile technique, a calculated volume of PlasmaLyte was removed and placed in a transfer pack.
6. The calculated volume of albumin was removed and the volume added to the PlasmaLyte.
7. The bag was mixed well, placed in a tube on ice and solution was allowed to chill for at least 10 minutes

B. Thawing and Washing
1. The exterior of the cryovial containing the hMSCs was wiped with 70% alcohol and placed in a bucket with ice.
2. Each vial was thawed one at a time
3. The vial was wiped down with 70% alcohol and place in the biological safety cabinet.
4. Using a 5 mL serological pipette thawed product was removed and place in the labeled “Thaw and Washed Product” tube.
5. Using an appropriate sized serological pipette the required amount of wash solution was removed (vial volume times 4).
   a. The wash solution was slowly added drop wise to the thawed product. The wash solution was gradually introduced to the cells while gently rinsing the product to allow the cells to adjust to normal osmotic conditions. Slow addition of wash solution with gentle agitation prevents cell membrane rupture from osmotic shock during thaw.
   b. 1 mL of the wash solution was used to rinse the cryovial.
   c. The rinse was added to the product conical tube.
6. The conical tube was placed on ice.
7. Steps 1-5 were repeated for any remaining vials.
   a. For higher doses the volume was split in half, with one half of the volume thawed in one 250 mL conical tube and the other half in the other 250 mL conical tube.
8. The Thaw and Washed Product tube was centrifuged at 500 g for 5 min. with the brake on slow.
9. A serological pipette was used to slowly remove the supernatant (approximately one inch from the cell pellet).
10. The cell pellet was resuspended in 5 mL of wash solution.
   a. For higher doses
      1) The cell pellets were resuspended in the remaining supernatant
      2) The cell pellets were combined.
      3) 5 mL of wash solution was used to rinse the conical tube in which the cell pellet was removed and add wash solution to the product.

### REFERENCES

Lange et al., Accelerated and safe expansion of human mesenchymal stem cells in animal serum-free medium for transplantation and regenerative medicine. J. Cell. Physiol. 213:18-26, 2007.
Zarjou et al., Paracrine effects of mesenchymal stem cells in cisplatin-induced renal injury require heme oxygenase-1, Am J Physiol Renal Physiol 300:F254-F262 (2011).
Ferenbach et al., Hemeoxygenase-1 and Renal Ischaemia-Reperfusion Injury, Nephron Exp Nephrol 115:e33-e37 (2010).
Sikorski et al., The story so far: molecular regulation of the heme oxygenase-1 gene in renal injury, Am J Physiol Renal Physiol 286:F425-F441 (2004).
Ankrum et al., Mesenchymal stem cell therapy: Two steps forward, one step back. Trends Mol. Med. 16(5):203-209 (2010).
Hare et al., A Randomized, Double-Blind, Placebo-Controlled, Dose-Escalation Study of Intravenous Adult Human Mesenchymal Cells (Prochymal) after Acute Myocardial Infarction. J Am Coll Cardiol. 54:2227-2286 (2009).
Cao et al., Mesenchymal stem cells derived from human umbilical cord ameliorate ischemia/reperfusion-induced acute renal failure in rats. Biotechnol Lett. 32:725-732 (2010).
Tögel et al., Autologous and allogeneic marrow stromal cells are safe and effective for the treatment of acute kidney injury. Stem Cells Dev. 18:475-485 (2009).
Kebriaei et al., Adult Human Mesenchymal Stem Cells Added to Corticosteroid Therapy for the Treatment of Acute Graft-versus-Host Disease. Biol Blood Marrow Transplant. 15:804-811 (2009).
Exner et al., The Role of Heme Oxygenase-1 Promoter Polymorphisms in Human Disease. Free Radical Biology & Medicine 37(8):1097-104 (2004).
Lange et al., Platelet Lysate for Rapid Expansion of Human Mesenchymal Stromal Cells. Cellular Therapy and Transplanation 1:49-53 (2008).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of generating a population of human mesenchymal stromal cells (MSCs), the method comprising:
(a) obtaining human MSCs; and
(b) determining the number of GT repeats present in the heme oxygenase-1 (HO-1) promoter region of both alleles.

2. The method of claim 1, further comprising the step of:
(c) selecting those human MSCs having 32 or fewer GT repeats in both alleles.

3. The method of claim 1 or claim 2, further comprising the step of expanding the human MSCs in a platelet lysate supplemented culture medium, to generate an expanded population of human MSCs.

4. The method of claim 3, wherein the human MSCs are expanded prior to determining the number of GT repeats present in both alleles.

5. The method of claim 3, wherein the number of GT repeats present in both alleles is determined prior to expanding the human MSCs.

6. A method of assaying the therapeutic effectiveness of human mesenchymal stromal cells (MSCs) for treating a pathology in a subject comprising:
(a) obtaining a population of human MSCs; and
(b) analyzing the number of GT repeats present in the heme oxygenase-1 (HO-1) promoter region of both alleles to determine whether the MSCs have short, medium, or long alleles, wherein the presence of two short alleles, two medium alleles, or one short allele and one medium allele indicates that the population contains MSCs that are therapeutically effective.

7. The method of claim 6, wherein the population of human MSCs is autologous to the subject.

8. The method of claim 6, wherein the population of human MSCs is allogeneic to the subject.

9. The method of claim 6, wherein the population of human MSCs is obtained from a cryopreserved sample.

10. The method of claim 6, wherein the population of human MSCs is obtained from a Master Cell Bank (MCB).

11. The method of claim 6, wherein the population of human MSCs is obtained from a bone marrow sample.

12. The method of claim 6, wherein the pathology is selected from the group consisting of a neurological pathology, an inflammatory pathology, a renal pathology, a hepatic pathology, a cardiovascular pathology, a retinal pathology, a muscular pathology, a bone-related pathology, a gastrointestinal pathology, a skin related pathology and a metabolic pathology.

13. The method of claim 12, wherein the renal pathology is selected from the group consisting of acute kidney injury, acute renal failure, chronic renal failure, chronic kidney disease, transplant, diabetic nephropathy, and hypertensive nephropathy.

14. The method of claim 12, wherein the neurological pathology is stroke.

15. The method of claim 12, wherein the inflammatory pathology is multi-organ failure.

16. The method of claim 12, wherein the metabolic pathology is diabetes.

17. The method of claim 6, wherein the short allele has ≦26 GT repeats.

18. The method of claim 6, wherein the medium allele has between 27 and 32 GT repeats.

19. The method of claim 6, wherein the long allele has >32 GT repeats.

20. The method of claim 6, wherein the number of GT repeats is analyzed using Fragment Length Analysis.

21. A method of selecting donors having therapeutically effective human mesenchymal stromal cells (MSCs) for treating a pathology in a subject comprising:
(a) analyzing the number of GT repeats present in the heme oxygenase-1 (HO-1) promoter region of both alleles of a potential human bone marrow donor to determine whether the potential donors has short, medium, or long alleles, wherein the presence of two short alleles, two medium alleles, or one short allele and one medium allele indicates that the potential donor would provide MSCs that are superior for therapeutic uses, and
(b) selecting those donors having such MSCs.

22. The method of claim 21, wherein the donor is autologous to the subject

23. The method of claim 21, wherein the donor is allogeneic to the subject.

24. The method of claim 21, wherein the number of GT repeats is analyzed from a blood sample.

25. The method of claim 21, wherein the wherein the number of GT repeats is analyzed from a cryopreserved sample.

26. The method of claim 21, wherein the wherein the number of GT repeats is analyzed from a sample from a Master Cell Bank (MCB).

27. The method of claim 21, wherein the number of GT repeats is analyzed from a bone marrow sample.

28. The method of claim 21, wherein the number of GT repeats is analyzed from other genetic material.

29. The method of claim 21, wherein the pathology is selected from the group consisting of a neurological pathology, an inflammatory pathology, a renal pathology, a hepatic pathology, a cardiovascular pathology, a retinal pathology, a muscular pathology, a bone-related pathology, a gastrointestinal pathology, a skin related pathology and a metabolic pathology.

30. The method of claim 29, wherein the renal pathology is selected from the group consisting of acute kidney injury, acute renal failure, chronic renal failure, chronic kidney disease, transplant, diabetic nephropathy, and hypertensive nephropathy.

31. The method of claim 29, wherein the neurological pathology is stroke.

32. The method of claim 29, wherein the inflammatory pathology is multi-organ failure.

33. The method of claim 29, wherein the metabolic pathology is diabetes.

34. The method of claim 21, wherein the short allele has ≦26 GT repeats.

35. The method of claim 21, wherein the medium allele has between 27 and 32 GT repeats.

36. The method of claim 21, wherein the long allele has >32 GT repeats.

37. The method of claim 21, wherein the number of GT repeats is analyzed using Fragment Length Analysis

38. A method of treating an MSC-related pathology in a subject in need thereof comprising:
(a) obtaining a population of human MSCs;
(b) analyzing the number of GT repeats present in the heme oxygenase-1 (HO-1) promoter region of both alleles to determine whether the MSCs have short, medium, or long alleles, wherein the presence of two short alleles, two medium alleles, or one short allele and one medium allele indicates that the population contains MSCs that are therapeutically effective; and
(c) administering any effective dose of the therapeutically effective MSCs to the subject, thereby treating the MSC-related pathology in the subject.

39. The method of claim 38, wherein the population of human MSCs is autologous to the subject.

40. The method of claim 38 wherein the population of human MSCs is allogeneic to the subject.

41. The method of claim 38, wherein the population of human MSCs is obtained from a cryopreserved sample.

42. The method of claim 38, wherein the population of human MSCs is obtained from a Master Cell Bank (MCB).

43. The method of claim 38, wherein the population of human MSCs is obtained from a bone marrow sample.

44. The method of claim 38, wherein the MSC-related pathology is selected from the group consisting of a neurological pathology, an inflammatory pathology, a renal pathology, a hepatic pathology, a cardiovascular pathology, a retinal pathology, a muscular pathology, a bone-related pathology, a gastrointestinal pathology, a skin-related pathology and a metabolic pathology.

45. The method of claim 44, wherein the renal pathology is selected from the group consisting of acute kidney injury, acute renal failure, chronic renal failure, chronic kidney disease, transplant, diabetic nephropathy, and hypertensive nephropathy.

46. The method of claim 44, wherein the neurological pathology is stroke.

47. The method of claim 44, wherein the inflammatory pathology is multi-organ failure.

48. The method of claim 44, wherein the metabolic pathology is diabetes.

49. The method of claim 44, wherein the short allele has ≦26 GT repeats.

50. The method of claim 44, wherein the medium allele has between 27 and 32 GT repeats.

51. The method of claim 44, wherein the long allele has >32 GT repeats.

52. A method of treating an MSC-related pathology in a subject in need thereof comprising:
(a) analyzing the number of GT repeats present in the heme oxygenase-1 (HO-1) promoter region of both alleles of a potential human donor to determine whether the potential donor has short, medium, or long alleles, wherein the presence of two short alleles, two medium alleles, or one short allele and one medium allele indicates that the potential donor would provide MSCs that are superior for therapeutic uses;
(b) selecting those donors having such MSCs; and
(c) administering any effective dose of the therapeutically effective MSCs to the subject, thereby treating the MSC-related pathology in the subject.

53. The method of claim 52, wherein the donor is autologous to the subject.

54. The method of claim 52, wherein the donor is allogeneic to the subject.

55. The method of claim 52, wherein the number of GT repeats is analyzed from a blood sample.

56. The method of claim 52, wherein the number of GT repeats is analyzed from a cryopreserved sample.

57. The method of claim 52, wherein the number of GT repeats is analyzed from a sample from a Master Cell Bank (MCB).

58. The method of claim 52, wherein the number of GT repeats is analyzed from a bone marrow sample.

59. The method of claim 52 wherein the MSC-related pathology is selected from the group consisting of a neurological pathology, an inflammatory pathology, a renal pathology, a hepatic pathology, a cardiovascular pathology, a retinal pathology, a muscular pathology, a bone-related pathology, a gastrointestinal pathology, a skin-related pathology and a metabolic pathology.

60. The method of claim 59, wherein the renal pathology is selected from the group consisting of acute kidney injury, acute renal failure, chronic renal failure, chronic kidney disease, transplant, diabetic nephropathy, and hypertensive nephropathy.

61. The method of claim 59, wherein the neurological pathology is stroke.

62. The method of claim 59, wherein the inflammatory pathology is multi-organ failure.

63. The method of claim 59, wherein the metabolic pathology is diabetes.

64. The method of claim 52, wherein the short allele has ≦26 GT repeats.

65. The method of claim 52, wherein the medium allele has between 27 and 32 GT repeats.

66. The method of claim 52, wherein the long allele has >32 GT repeats.

67. A kit comprising reagents for the analyzing the number of GT repeats present in the HO-1 promoter region of both alleles in a population of human MSCs.

68. The kit of claim 67, wherein the reagents for analyzing the number of GT repeats comprise reagents for use in Fragment Length Analysis.

69. The kit of claim 67, wherein the reagents for analyzing the number of GT repeats comprise reagents for use with polymerase chain reaction (PCR).

70. A method of producing a dosage form of therapeutically effective human MSCs comprising:
(a) obtaining a population of human MSCs;
(b) analyzing the number of GT repeats present in the heme oxygenase-1 (HO-1) promoter region of both alleles to determine whether the MSCs have short, medium, or long alleles, wherein the presence of two short alleles, two medium alleles, or one short allele and one medium allele indicates that the population contains MSCs that are therapeutically effective, and
(c) selecting therapeutically effective human MSCs.
thereby producing a dosage form of human MSCs.

71. The method of claim 70, wherein the population of human MSCs is autologous to the subject.

72. The method of claim 70, wherein the population of human MSCs is allogeneic to the subject.

73. The method of claim 70, wherein the population of human MSCs is obtained from a cryopreserved sample.

74. The method of claim 70, wherein the population of human MSCs is obtained from a Master Cell Bank (MCB).

75. The method of claim 70, wherein the population of human MSCs is obtained from a bone marrow sample.

76. The method of claim 70, wherein the short allele has ≦26 GT repeats.

77. The method of claim 70, wherein the medium allele has between 27 and 32 GT repeats.

78. The method of claim 70, wherein the long allele has >32 GT repeats.

79. A method of producing a dosage form of therapeutically effective MSCs comprising:
(a) analyzing the number of GT repeats present in the heme oxygenase-1 (HO-1) promoter region of both alleles of a potential human donor to determine whether the potential donor has short, medium, or long alleles, wherein the presence of two short alleles, two medium alleles, or one short allele and one medium allele indicates that the potential donor would provide MSCs that are superior for therapeutic uses, and
(b) selecting those donors having such MSCs
thereby producing a dosage form of human MSCs.

80. The method of claim 79, wherein the donor autologous to the subject.

81. The method of claim 79, wherein the donor is allogeneic to the subject.

82. The method of claim 79, wherein the number of GT repeats is analyzed from a blood sample.

83. The method of claim 79, wherein the number of GT repeats is analyzed from a cryopreserved sample.

84. The method of claim 79, wherein the number of GT repeats is analyzed a sample from a Master Cell Bank (MCB).

85. The method of claim 79, wherein the number of GT repeats is analyzed from a bone marrow sample.

86. The method of claim 79, wherein the short allele has ≦26 GT repeats.

87. The method of claim 79, wherein the medium allele has between 27 and 32 GT repeats.

88. The method of claim 79, wherein the long allele has >32 GT repeats.

89. A population of human MSCs, wherein the human MSCs in the population contain ≦32 GT repeats in each allele of the HO-1 promoter region.

90. The population of claim 89, wherein the human MSCs in the population contain two short alleles, two medium alleles, or one short allele and one medium allele of the HO-1 promoter region.

91. The population of claim 90, wherein the population of human MSCs has been cultured in platelet lysate supplemented culture media.

92. The population of claim 90, wherein the population of human MSCs expresses Prickle 1 at a higher degree than MSCs that have been cultured in fetal calf serum supplemented culture media.

93. The population of claim 90, wherein the population of human MSCs expresses Prickle 1 to an eight-fold higher degree than MSCs that have been cultured in fetal calf serum supplemented culture media.

94. The population of claim 90, wherein the population of human MSCs that have been cultured in platelet lysate are less immunogenic than MSCs that have been cultured in fetal calf serum supplemented culture media.

95. A population of human MSCs comprising at least 75% human MSCs, wherein:
a) the human MSCs in the population contain ≦32 GT repeats in each allele of the HO-1 promoter region;
b) the human MSCs in the population have been in platelet lysate supplemented culture media and express Prickle 1 at a higher degree than MSCs that have been cultured in fetal calf serum supplemented culture media;
c) the human MSCs are cultured to between 80 and 95% confluence;
d) the population does not contain detectable levels of infectious agents; and
e) the human MSCs in the population have only undergone fewer than 30 population doublings.

96. A method for treating a pathology in a subject comprising administering a therapeutically effective amount of the population of human MSCs of claim 89 or claim 95 to the subject.

97. The method of claim 96, wherein the population of human MSCs is autologous to the subject.

98. The method of claim 96, wherein the population of human MSCs is allogeneic to the subject.

99. The method of claim 96, wherein the pathology is selected from the group consisting of a neurological pathology, an inflammatory pathology, a renal pathology, a hepatic pathology, a cardiovascular pathology, a retinal pathology, a muscular pathology, a bone-related pathology, a gastrointestinal pathology, a skin related pathology and a metabolic pathology.

100. The method of claim 99, wherein the renal pathology is selected from the group consisting of acute kidney injury, acute renal failure, chronic renal failure, chronic kidney disease and transplant.

101. The method of claim 99, wherein the neurological pathology is stroke.

102. The method of claim 99, wherein the inflammatory pathology is multi-organ failure.

103. The method of claim 99, wherein the metabolic pathology is diabetes.
